(19) United States
(12) Patent Application Publication
(10) Pub. No.: US 2008/0228193 A1
(43) Pub. Date: Sep. 18, 2008

(54) IMPLANTABLE MEDICAMENT DELIVERY DEVICE AND DELIVERY TOOL AND METHOD FOR USE THEREWITH

(75) Inventor: Amir M. MATITYAHU, Los Altos, CA (US)

Correspondence Address: Dorsey & Whitney LLP;US Bank Center, 1420 Fifth Avenue, Suite 3400, Seattle, WA, 98101-4010, US

(73) Assignee: ANTHEM ORTHOPAEDICS LLC, Los Altos, CA (US)

(21) Appl. No.: 12/044,955

(22) Filed: Mar. 8, 2008

Related U.S. Application Data
(60) Provisional application No. 60/906,092, filed on Mar. 9, 2007.

(51) Int. Cl. A61B 17/88; A61K 9/00

(52) U.S. Cl. 606/99; 424/422

(57) ABSTRACT

An implantable medicament delivery device is disclosed for administration of a medicament to a joint. The implantable delivery device includes a filament formed of a bioabsorbable material and carrying a medicament. The material of the filament is capable of eluting the medicament. A kit for use in post operatively treating a joint of a mammalian body is also disclosed. The kit comprises a package including the bioabsorbable filament carried within the package, which bioabsorbable filament carries a medicament. Delivery tools and methods are also disclosed for implantation of the medicament delivery device.

### RELATED APPLICATION DATA

This application claims benefit of provisional application Ser. No. 60/906,092 filed Mar. 9, 2007, the entire content of which is expressly incorporated by reference herein.

### FIELD OF THE INVENTION

In general, invention relates to the delivery of a medicament to a target area of a mammalian body and, more particularly, to the local delivery of the medicament to the target area.

### BACKGROUND

It is a common practice after percutaneous and arthroscopic procedures that the patient is typically sent home and responsible for maintaining a pain management regimen. It is known that physical therapy is critical for proper healing and range of motion of the repaired joint by percutaneous or arthroscopic procedures. Effective pain management allows the patient to comfortably perform the required exercises. Current pain management treatment includes oral pain medications, intravenous pain medications and intra-articular infusion of anesthetic or analgesic agents. When local intra-articular infusion of analgesics is used for the treatment of post operative pain relief, the patient needs less oral and/or intravenous medications.

Typically, however, the infusion of analgesics after arthroscopic procedures require the placement of a catheter into the articular space or region and then infusion of analgesics and/or anesthetics from a metered reservoir for a period of, for example, three days post-operatively, after which, the physician must carefully remove the catheter at a follow up appointment. Alternatively, a drug-coated catheter can be placed directly into the articular space. The catheter is coated with analgesic or anesthetic medication that will elute over, for example, a three to five day period. This catheter also has to be removed by the physician.

Several disadvantages arise with these current drug delivery systems and treatment methods. For example, metered drugs systems are expensive, complicated and necessitate that the patient carries the reservoir with them for the prescribed time period. Additionally, the metered pump may not provide enough medication or, alternatively, may administer the medication too quickly. In the aforementioned procedures, the catheter itself also potentially increases the risk of infection by providing a pathway from outside of the body through the skin and into the joint space. As described previously, the physician must eventually remove the catheter once the treatment is complete, which requires an additional appointment.

In view of the foregoing, a need exists in the art for a device and method of implantation of such device which includes a medicament for treating a patient. A need also exists for such a device and method that provides secure placement and anchoring of the device for localized delivery of a medicament into the target area of the mammalian body.

### SUMMARY OF THE INVENTION

In the invention disclosed, an implantable medicament delivery device can be provided for administration of a medicament to mammalian body. The implantable delivery device includes an implantable filament formed of a bioabsorbable material and carrying a medicament. The material of the filament is capable of eluting the medicament. A kit for use in post operatively treating a joint of a mammalian body is also provided. The kit comprises a package including the bioabsorbable filament carried within the package, which bioabsorbable filament carries a medicament.

A delivery tool for use with an implantable device to treat a joint of a mammalian body can also be provided. The tool is formed of an elongate tubular member having a proximal end and a distal end and a passageway extending from the proximal end to the distal end. A penetration element having a sharpened tip is slidably disposed in the passageway and moveable between a first position in which the tip is recessed within the distal end of the elongate tubular member and a second position in which the tip is at least partially extended from the distal end. The penetration element is adapted to carry the implantable device. The delivery tool also includes an actuation mechanism which is at least partially carried by the elongate member for moving the penetration element from the first position to the second and for delivering the implantation device from the elongate tubular member into an implanted position in the joint. A method of administering a medication to a mammalian body is also provide. The method includes the step of implanting a filament into a joint of the mammalian body. The filament is formed of a bioabsorbable material and carries a medicament. The method further includes the step of eluting the medicament from the filament after placement of the filament in the joint to aid in healing of the mammalian body in the vicinity of the joint.

Other features of the present invention will become apparent from the following description along with the accompanying drawings.

### BRIEF DESCRIPTION OF THE DRAWINGS

FIG. 1a is an isometric view of a first embodiment of a bioabsorbable filament implant.

FIG. 1b is a partial side view of a first embodiment of a bioabsorbable filament implant.

FIG. 1c is a front view of a first embodiment of a bioabsorbable filament implant showing the concentricity of the implant.

FIG. 2a is an off-axis side view of a second embodiment of a bioabsorbable filament implant that shows recessed features.

FIG. 2b is a top-view of a second embodiment of a bioabsorbable filament implant.

FIG. 2c is a cross-section side view of FIG. 2b, taken along the line 2c-2c of FIG. 2b, that shows the recessed features.

FIG. 3a is a top view of a third embodiment of a bioabsorbable filament implant.

FIG. 3b is a cross-sectional side view of FIG. 3a, taken along the line 3b-3b of FIG. 3a, showing features within the tip.

FIG. 3c is an end view showing the shape of the interface feature.

FIG. 4 is an isometric view of an embodiment of the invention that shows the boss feature for interfacing with delivery tools.

FIGS. 5a-5f are isometric views that show variations of the tip designs or anchor that incorporate ridges, conical features, and flexible tabs.

FIGS. 6a-6e are isometric views that show variations in design/shape of the elongated filament member.

FIG. 7 is an isometric view that shows a tip design with another type boss feature.

FIG. 8a is an isometric view of the first embodiment of the bioabsorbable filament implant mounted on a delivery tip of a delivery tool.

FIG. 8b is an isometric view of the first embodiment of the bioabsorbable filament implant after the delivery tip is retracted.

FIG. 9 is an isometric view of the first embodiment of the bioabsorbable filament implant showing the slight inward bias of the delivery tip from the two slots to provide added friction, as the delivery tip is retracted.

FIG. 10a is an isometric view of the embodiment of the bioabsorbable filament implant shown in FIG. 3a mounted on a delivery tip.

FIG. 10b is an isometric view of the embodiment of the bioabsorbable filament implant shown in FIG. 3a as the delivery tip is retracted.

FIG. 10c is an isometric view of the embodiment of the bioabsorbable filament implant shown in FIG. 3a as it is deployed from the delivery tip by a pushrod.

FIG. 11 is an isometric view of the embodiment of the bioabsorbable filament implant shown in FIG. 3a as it is deployed from the delivery tip by a pushrod, with added friction from the slight outward bias of the delivery tips.

FIG. 12 is an isometric view of the bioabsorbable filament implant shown in FIG. 4 as delivery tip is retracted, with added friction from the slight inward bias of the delivery tips.

FIG. 13a is an isometric view of the delivery tool of FIG. 8a.

FIG. 13b is an isometric view of certain internal components of the delivery tool of FIG. 13a.

FIG. 14a is a partial cut-away isometric view of the delivery tool of FIG. 13a.

FIG. 14b is a partial cut-away isometric view of the certain internal components of FIG. 13b.

FIG. 14c is a cross-section side view of the tip of the delivery tool of FIG. 13a taken along the line 14c-14c of FIG. 14d.

FIG. 14d is a top view of the tip of the delivery tool of FIG. 13a.

FIG. 15a is a partial cut-away isometric view of the delivery tool of FIG. 13a, as the handle is actuated and the pointed penetrating tip is deployed.

FIG. 15b is a partial cut-away isometric view of certain internal components of FIG. 13b, as the handle is actuated and the pointed penetrating tip is deployed.

FIG. 15c is a cross-section side view of the tip of delivery tool of FIG. 13a, taken along the line 15c-15c of FIG. 15d, showing the pointed penetrating tip deployed as the handle is actuated.

FIG. 15d is a top view of the tip of the delivery tool of FIG. 13a, showing the pointed penetrating tip deployed as the handle is actuated.

FIG. 16a is a partial cut-away isometric view of the delivery tool of FIG. 13a, as the handle is further actuated and the pointed penetrating tip is retracted.

FIG. 16b is a partial cut-away isometric view of certain internal components of FIG. 13b, as the handle is further actuated and the pointed penetrating tip is retracted.

FIG. 16c is a cross-section side view of the tip of the delivery tool of FIG. 13a, taken along the line 16c-16c of FIG. 16d, showing the pointed penetrating tip retracted as the handle is further actuated.

FIG. 16d is a top view of the tip of the delivery tool of FIG. 13a, showing the pointed penetrating tip retracted as the handle is further actuated.

FIG. 17a is a partial cut-away isometric view of the delivery tool of FIG. 13a, as the handle is fully actuated and the tip of the bioabsorbable filament implant is deployed.

FIG. 17b is a partial cut-away isometric view of certain internal components of the delivery tool of FIG. 13a, as the handle is fully actuated and the tip of the bioabsorbable filament implant is deployed.

FIG. 17c is a cross-section side view of the tip of the delivery tool of FIG. 13a, taken along the line 17c-17c of FIG. 17d, as the handle is fully actuated and the tip of the bioabsorbable filament implant is deployed.

FIG. 17d is a top view of the tip of the delivery tool of FIG. 13a, showing the tip of the bioabsorbable filament implant deployed as the handle is fully actuated.

FIG. 18a is a partial cut-away isometric view of the delivery tool of FIG. 13a as the delivery tool is retracted and the bioabsorbable filament implant is released.

FIG. 18b is a partial cut-away isometric view of certain internal components of the delivery tool of FIG. 13a as the delivery tool is retracted and the bioabsorbable filament implant is released.

FIG. 18c is a cross-section side view of the tip of the delivery tool of FIG. 13a, taken along the line 18c-18c of FIG. 18d, as the delivery tool is retracted and the bioabsorbable filament implant is released.

FIG. 18d is a top view of the tip of the delivery tool of FIG. 13a, showing the bioabsorbable filament implant being released as the delivery tool is retracted.

FIG. 19 is an isometric view of the bioabsorbable filament implant with tail.

FIG. 20 is an isometric view of the bioabsorbable filament implant with tail and tail catch.

FIG. 21 is an isometric view of the distal tip of the tool showing compression mechanism and bioabsorbable filament implant of FIG. 19 connected.

FIG. 22a is a cross-section side view of the distal tip of the tool of FIG. 21, taken along the line 22a-22a of FIG. 22b, showing the compression mechanism and bioabsorbable filament implant connected.

FIG. 22b is a top view of the distal tip of the tool of FIG. 21, showing the compression mechanism and bioabsorbable filament implant connected.

FIG. 22c is a side view of the distal tip of the tool of FIG. 21, showing the compression mechanism and bioabsorbable filament implant connected.

FIG. 23a is a cross-section side view of the distal tip of the tool of FIG. 21, taken along the line 23a-23a of FIG. 23b, showing the slidable proximal jaw retracted that releases the compression mechanism.

FIG. 23b is a top view of the distal tip of the tool of FIG. 21, showing the slidable proximal jaw retracted that releases the compression mechanism.

FIG. 23c is a side view of the distal tip of the tool of FIG. 21, showing the slidable proximal jaw retracted that releases the compression mechanism.

FIG. 24a is a cross-section side view of the distal tip of the tool of FIG. 21, taken along the line 24a-24a of FIG. 24b, showing the release spring deflect to help expand the filament member.

FIG. 24b is a top view of the distal tip of the tool of FIG. 21, showing the release spring deflect to help expand the filament member.

FIG. 24c is a side view of the distal tip of the tool of FIG. 21, showing the release spring deflect to help expand the filament member.

FIG. 25a is a cross-section side view of the distal tip of the tool of FIG. 21, taken along the line 25a-25a of FIG. 25b, showing the bioabsorbable filament implant as it rotates to release.

FIG. 25b is a top view of the distal tip of the tool of FIG. 21, showing the bioabsorbable filament implant as it rotates to release.

FIG. 25c is a side view of the distal tip of the tool of FIG. 21, showing the bioabsorbable filament implant as it rotates to release.

FIG. 26a is a cross-section side view, of the distal tip of the tool of FIG. 21, taken along the line 26a-26a of FIG. 21, showing the bioabsorbable filament implant as it released fully from the tool.

FIG. 26b is a top view of the distal tip of the tool of FIG. 21, showing the bioabsorbable filament implant as it released fully from the tool.

FIG. 26c is a side view of the distal tip of the tool of FIG. 21, showing the bioabsorbable filament implant as it released fully from the tool.

FIG. 27 is an isometric view of an implant tip with elongated boss feature with cross-hole.

FIG. 28 is an isometric view of an alternative embodiment of an implant tip with pin-like boss feature with cross-hole.

FIG. 29 is an isometric view of an implant tip of FIG. 27, showing a filament element fitted to the cross-hole.

FIG. 30 is an isometric view of the implant tip of FIG. 28, showing a filament element fitted to the cross-hole.

FIG. 31 is a cross-sectional anterior-posterior view of a human shoulder with an arthroscopic port.

FIG. 32 is a cross-sectional anterior-posterior view of a human shoulder with an arthroscopic port of FIG. 31, showing a delivery tool, the support tube of the delivery tool being firmly approximated to the capsule and scapula of the shoulder.

FIG. 33 is a cross-sectional anterior-posterior view of a human shoulder with an arthroscopic port with delivery tool of FIG. 32, where the penetrating tips of the delivery tool puncture and penetrate the underlying tissue/bone when the handle is actuated.

FIG. 34 is a cross-sectional anterior-posterior view of a human shoulder with an arthroscopic port with delivery tool of FIG. 32, where the handle of the delivery tool is fully actuated to completely drive the tip of the implant into the tissue/bone.

FIG. 35 is a cross-sectional anterior-posterior view of a human shoulder with an arthroscopic port with delivery tool of FIG. 32, where the delivery tool is retracted.

FIG. 36 is a cross-sectional anterior-posterior view of a human shoulder with an arthroscopic port of FIG. 31, where the implant has been anchored within the capsule of the shoulder.

FIG. 37 is a cross-sectional medial-lateral view of a human knee with an arthroscopic port.

FIG. 38 is a cross-sectional medial-lateral view of a human knee with an arthroscopic port of FIG. 37, showing a delivery tool, the support tube of the delivery tool being firmly approximated to the capsule on either side of the patellar tendon.

FIG. 39 is a cross-sectional medial-lateral view of a human knee with an arthroscopic port with the delivery tool of FIG. 38, where the penetrating tips of the delivery tool puncture and penetrate the underlying tissue/bone when the handle is actuated.

FIG. 40 is a cross-sectional medial-lateral view of a human knee with an arthroscopic port with the delivery tool of FIG. 38, where the handle of the delivery tool is fully actuated to completely drive the tip of the implant into the tissue/bone.

FIG. 41 is a cross-sectional medial-lateral view of a human knee with an arthroscopic port with the delivery tool of FIG. 38, where the delivery tool is retracted.

FIG. 42 is a cross-sectional medial-lateral view of a human knee with an arthroscopic port of FIG. 37, where the implant has been anchored within the capsule of the knee.

FIG. 43a is an isometric view of a cannulated bioabsorbable filament implant adjacent to a wire.

FIG. 43b is an isometric view of the cannulated bioabsorbable filament implant of FIG. 43a guided over a wire.

FIG. 44a is a close-up, side view of a bioabsorbable filament implant, with a threaded tip fitted to a delivery tip.

FIG. 44b is a side view of a bioabsorbable filament implant, with a threaded tip fitted to a delivery tip of FIG. 44a that is mounted within a ribbed handle.

FIGS. 45a-45c shows an alternative handle mechanism for controlling the release of the implant.

FIG. 46 shows an exemplary embodiment of a kit containing a bioabsorbable filament implant of FIG. 4a and delivery tool of FIG. 13a.

### DETAILED DESCRIPTION OF PREFERRED EMBODIMENTS

The present invention is an implantable bioabsorbable filament that elutes a therapeutic compound near or within a target articular joint for a prescribed period of time and without the need to remove the implant, as it will be completely absorbed by the fluids within the tissue.

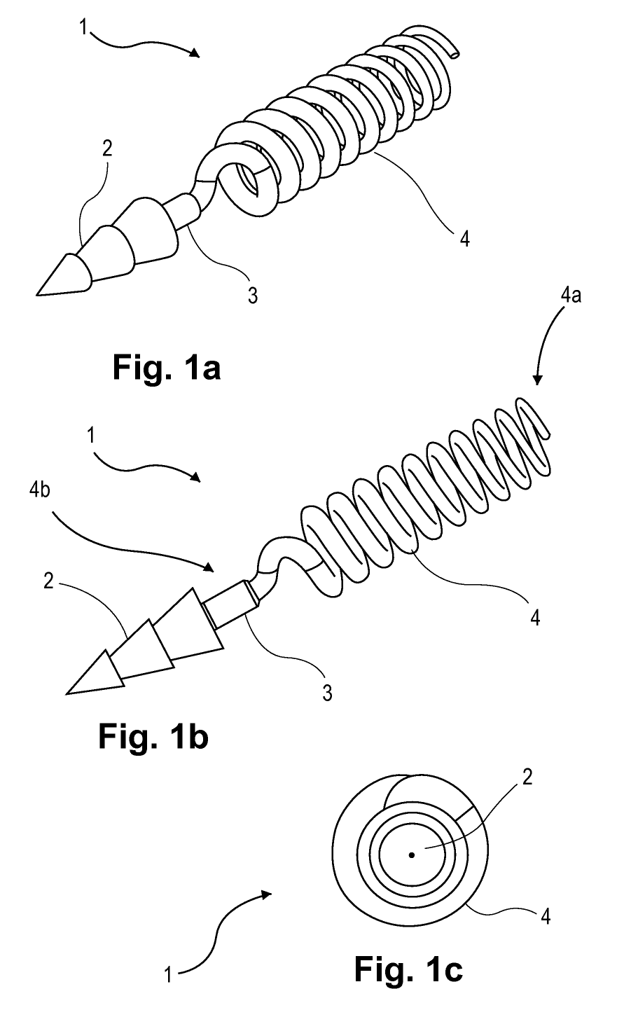

As depicted in the drawings, FIG. 1a shows a first embodiment of a bioabsorbable filament implant 1 for administration of a medicament to a joint. The implant or implantable medicament delivery device 1 generally includes a tip 2 or anchoring member, an interface feature 3, and an elongated filament member 4. The delivery device may be formed of a unitary member or may include one or more distinct components joined together. For example, the implant may include a tip 2 formed separately and which is attached to elongated filament member 4, or alternatively may include a unitary elongated filament member 4 having a tip 2 integral therewith. To this end, any number of tips and tip geometries may be used with the elongated filament member 4. Likewise, interface feature 3 may be integral with either the filament member or tip or a distinct component joined thereto. In a preferred embodiment, the filament 4 includes an end and an anchor at the end for localizing the filament to tissue being treated in the mammalian body, although the filament member 4 may be used without the anchor 2 thereon. In the exemplary embodiment, the tip 2 is shown as a barb-like or conical feature that can be used to penetrate and secure or anchor the implant in tissue, for example capsular tissue, cartilage, bone, muscle and fat. The interface feature 3 provides a mechanically robust region on the bioabsorbable filament implant 1 that can used for securing to a delivery tool (not shown) or for grasping with instruments, graspers, or other arthroscopic tools (not shown) that already exist in the typical operating suite.

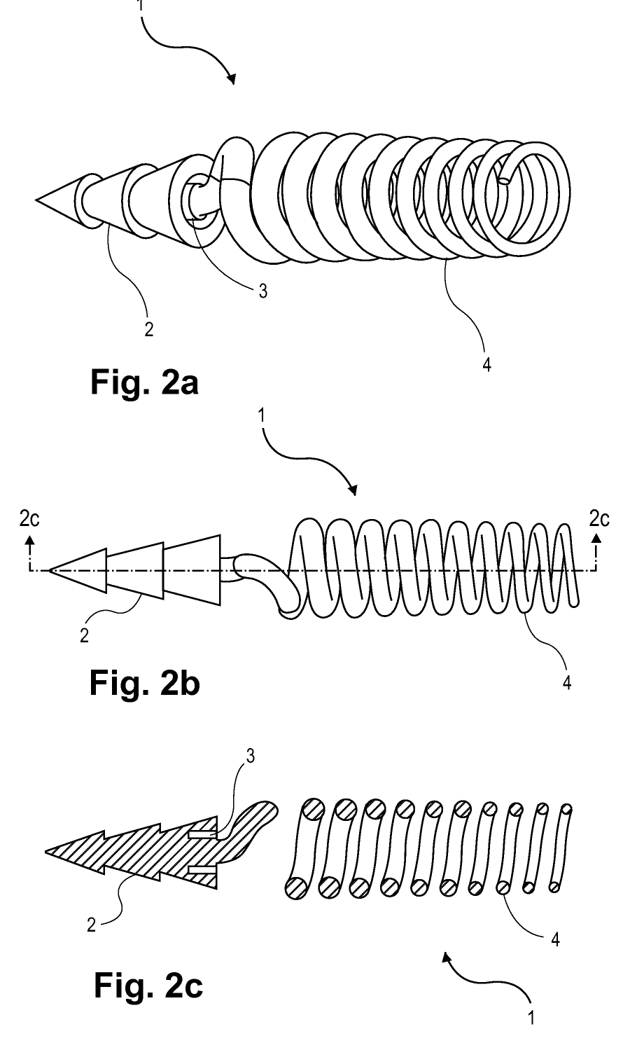

To provide an even more compact bioabsorbable filament implant 1 and to provide additional support when mounted to the hollow shaft of a delivery tip (not shown), the interface feature 3 can be recessed within the tip 2, as shown in FIG. 2a and FIG. 2b. The cross-section view 2c-2c in FIG. 2c, indicates the interface feature 3 within the tip 2, it being understood that the mechanical stability and strength can be significantly improved by engaging both the inner surface of the tip 2 and the outer surface of the interface feature 3 with a correspondingly fitting delivery tip (not shown).

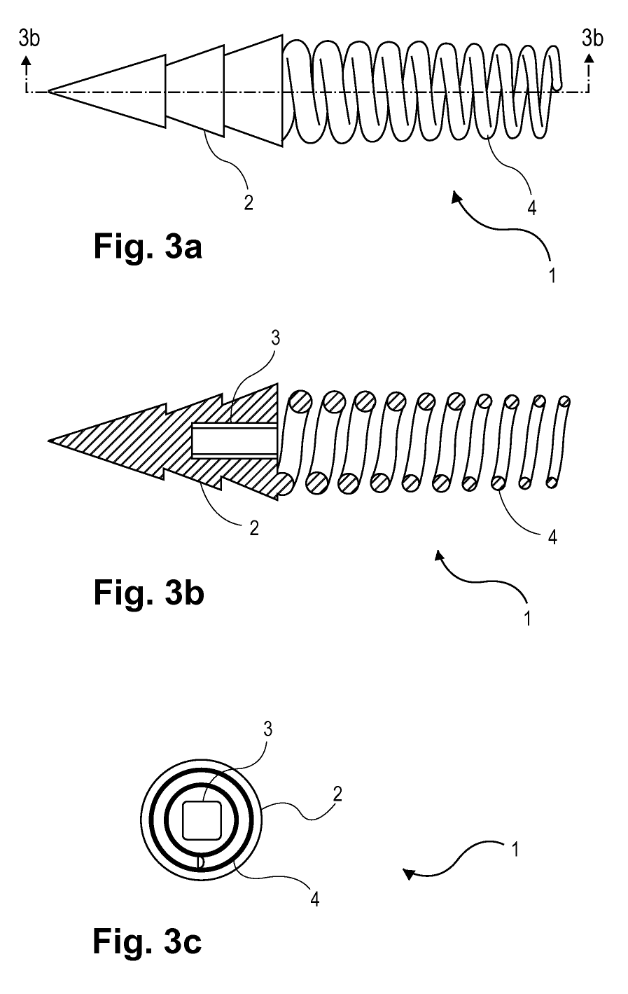

Furthermore, the interface feature 3 can be embedded entirely within the tip 2. The elongated filament member 4 can then be directly fitted to the tip 2 of the bioabsorbable filament implant 1, as depicted in FIG. 3a and cross-section view 3B-3B of FIG. 3b. This design further reduces the overall length of the device and also provides a more distributed attachment of the elongated filament member 4 to the tip 2, which may enhance the mechanical robustness of the attachment point. In addition, a non-central attachment point of the elongated filament member 4 to the tip 2 may be provided which allows for the delivery tip (not shown) to secure directly to the interface feature 3 within the tip 2. The interface feature 3 can be a square-drive, as shown in FIG. 3c, to prevent rotation about the delivery tip, or can have a circular, circle with a flat, oval, hexagonal, or any other suitable cross-section depending on whether the device should be free to rotate or not. The tip 2 can also be cannulated (not shown) to permit the bioabsorbable filament implant 1 to be delivered over a guidewire or over a small stainless drill guide along with a cannulated delivery tip (not shown).

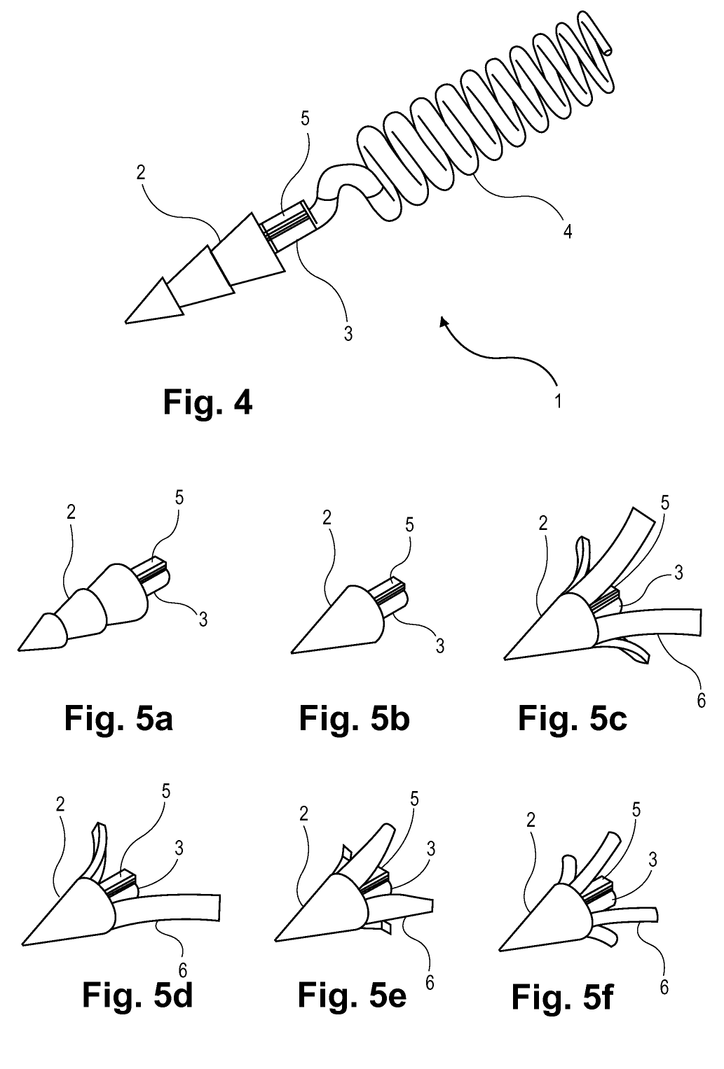

Alternatively, to provide anti-rotation and additional support when mounted to a delivery tip (not shown), an elongated boss feature 5, as shown in FIG. 4, can be integrated into the interface feature 3 of the bioabsorbable filament implant 1, like that depicted in FIG. 1a.

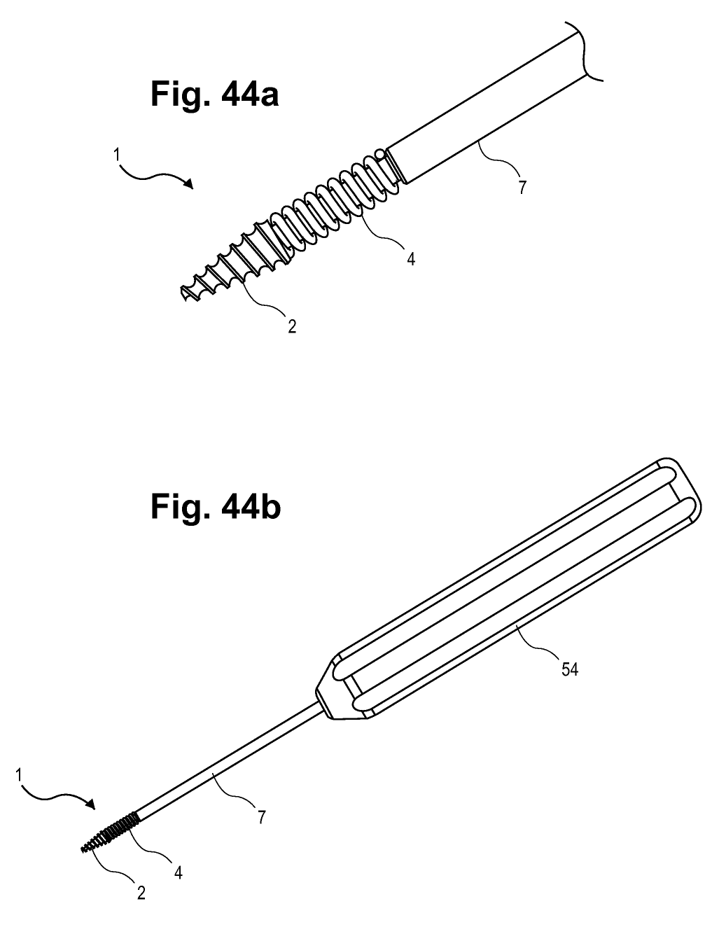

Generally, the tip 2 is formed or may include a generally tapered cone or barb. However, various tip 2 designs can be utilized to customize the retention force and overall size/profile of the distal end of the device. For instance, a single tapered cone or a plurality of tapered cones concentrically disposed along an axis and joined in series may be provided on the tip. The tip 2 length can be longer and anchor with more ridges, for instance a series of conical ridges or any suitable shape to keep the tip from dislodging. Preferably, the conical ridges may taper in diameter towards a tip as in FIG. 5a. Alternatively, as shown in FIG. 5b, a shorter tip 2 can be provided having fewer ridges for less retention. A plurality of flexible tabs 6, as shown in FIG. 5c, can be provided to increase retention within the tissue. Alternative arrangements may include fewer flexible tabs 6, as depicted in FIG. 5d, to provide less retention and a flatter profile. The shape of the flexible tabs 6 can also be tapered to provide customizable flex properties, as shown in FIG. 5e. The flexible tabs 6 can alternatively have different cross-sections, for example, circular, as depicted in FIG. 5f, square, oval, or any other suitable shape. As a non-limiting example, as can be seen from FIGS. 5c-5f a plurality of flexible tabs may be provided which extend proximally from a tapered cone in a circumferentially spaced-apart pattern. The tip may also include a threaded portion or be threaded to provide for threaded insertion of the bioabsorbable filament (as can be seen in FIG. 44a). The number, distribution, length, thickness, profile, and taper of the flexible tabs 6 and tip 2 can be designed in any suitable arrangement to provide the desirable flex and retention characteristics. Furthermore, while a “tip” is specifically described herein, any anchor or equivalent may be acceptable for use as described herein with the filament 4.

The filament 4 can simply be an elongated coil or can be further shaped and configured in order to minimize migration of the filament once placed within the patient. The filament may include a portion formed from an elongate member having a first end and a second end. One or both ends of the filament, in one embodiment, can be fashioned with a hook or other securement mechanism or feature which enables the filament to be secured to tissue, bone, cartilage, meniscus, or other bodily components within the articular space or other region in which the filament is placed. To this end, the filament 4 may also be securable or anchorable in its implanted position.

In this particular embodiment, the elongated filament member 4 may include a portion formed from an elongate member and having a diameter that decreases from the first end to the second end. The filament member 4 may be or include a coil-like or helical portion or feature that becomes progressively thinner in cross-section as the coil continues further from the tip 2, which is more clearly shown in FIG. 1b. To this end, the helical portion may be formed from an elongate member having a first end and a second end and a diameter that decreases from the first end to the second end. Preferably, the thickness or cross-section or diameter of the filament coil progressively decreases from an initial range of 0.0001 mm to 10 mm to a final cross-section ranging from 0.00001 mm to 10 mm. More preferably, the cross-section ranges from an initial 0.01 mm to 4 mm to a final cross-section of 0.1 mm to 2 mm In addition, the coil or helical feature of the filament may taper or narrow in successively smaller concentric circles from a first end to a second end. As shown in FIG. 1b, the filament tapers or increases in cross-section from its proximal end 4a to its distal end 4b. This design feature enables the bioabsorbable implant material to preferentially erode the thinnest portion of the elongated filament member 4 first and then propagate the absorption/erosion towards the thicker portion to ensure that the elongated filament member 4 does not prematurely erode from the tip 2 or interface feature 3 or within the middle section of the elongated filament member 4 and therefore prevents a portion of the implant from disengaging from the secured portion of the implant and circulating within the joint space. To minimize the profile and to enable the bioabsorbable filament implant 1 to be more easily inserted through an arthroscopic port, the tip 2 and the elongated filament member 4 are concentric about the long axis, as depicted in FIG. 1c.

Typical overall total lengths for the bioabsorbable filament implant 1 range from 0.25 inches to 10 inches and more preferably range from one inch to 4 inches, and even more preferably, may be approximately 0.5 inches. Overall diameter can range from 0.001 inches to 2 inches and more preferably range from 0.01 inches to 0.5 inches, and even more specifically range from 0.125 inches to 0.375 inches.

A filament, as described herein, may include a continuous object or elongate member, or cylindrical shaped member. The filament may include, but is not limited to, a thin flexible thread-like object or thread, a strip, strand, string, fiber, or wire. The filament may also be formed of a composite structure which is continuously wound, and/or may include fiber reinforcement. The filament can be made from any suitable bioabsorbable material or materials such as hydrophobic or hydrophilic polysaccharides or any suitable material that is biocompatible and bioabsorbable, or, for example, may be polylactic acid (PLA), polyglycolic acid (PGA) or combinations of PLA and PGA that provide the appropriate absorption rate, as understood in the art. For instance, the bioabsorbable material may consist of polylactic acid (PLA), polyglycolic acid (PGA), or combinations thereof to form co-polymers of PLA/PGA, also know as poly(lactide-co-glycolide). The bioabsorbable implant material can be impregnated, blended, coated, sprayed, contain micro-capsules, contain micro-spheres and/or be deposited with an analgesic, anesthetic, anti-inflammatory, steroid and/or other medicament, which is carried by the implant material and is eluted over a period of time, for example over a one to fourteen day period, or more preferably between three and five days. Preferably, the filament is impregnated with an analgesic, anesthetic, anti-inflammatory, steroid and/or other medicament, which is carried by the material of the filament and is eluted over a period of time, for example over a one to fourteen day period. The eluting material is preferably impregnated into the material of the filament in any suitable manner, but can also be coated or layered on the material of the filament or mixed in any suitable manner with the material of the filament. The analgesic concentration is sufficient to allow for pain relief that is maintained during the elusion phase. While specific geometries of the filament are described herein, alternative arrangements or combinations or equivalents suitable for the purposes provided herein would be acceptable for use with the present invention.

The bioabsorbable filament implant 1 can be machined, thermally formed, extruded, injection molded, or use any other manufacturing methods known in the art. Additionally, the bioabsorbable filament implant 1 can be assembled from individual components that are made using the previously mentioned processes and then fitted, press-fit, snap-fit, glued, RF welded, solvent bonded, and/or reflowed to create a single, finished bioabsorbable filament implant 1. The bioabsorbable implantable device can have a compliant free-form shape.

To further enhance the mechanical robustness of the bioabsorbable filament implant 1, a region of material near the tip 2, interface feature 3, and/or elongated filament member 4 with additional flexibility could help prevent possible fracture or fatigue due to excessive bending and flexing while implanted. This flexible region of the implant could be formed by using a material with a lower material modulus than the rest of the implant. This region could be introduced during the injection molding process by injecting materials with different material moduli into different regions of the implant part or by assembling separate components with different material moduli into one implant device by using methods described previously. The bioabsorbable implantable device and specifically the bioabsorbable material may thus contain regions of reduced material modulus to increase flexibility and minimize fatigue and fracture in high stress or high deflection regions.

Furthermore, the bioabsorbable filament implant 1 could have customized material stiffness along the entire device, by using materials with specific material properties in selected areas. For example, a relatively hard material could be used for the tip 2 to allow penetration into hard tissue and a relatively soft material could be used for the interface feature 3 to provide flexibility without failure and then a slightly stiffer material could used for the elongated filament member 4. Additionally, regions within the elongated filament member 4 can be introduced to provide regions of extra flexibility within the coils to enhance conformability. Materials with different bulk moduli could be created by blending the aforementioned materials to achieve the desirable material properties, as known in the art.

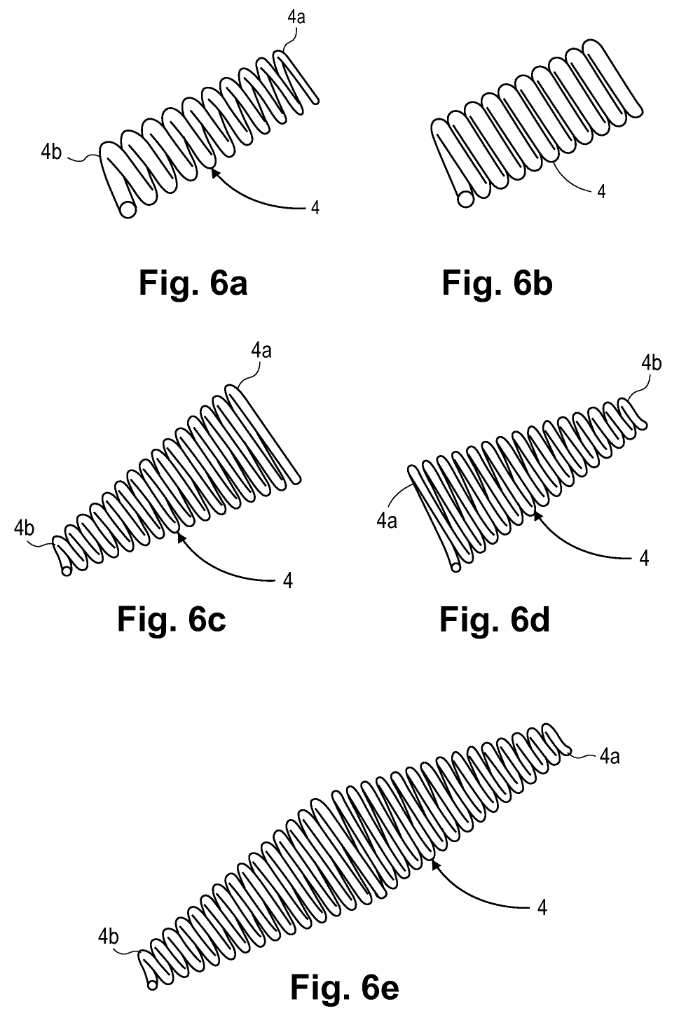

Accordingly, the elongated filament member 4 can have a filament cross-section that becomes progressively thinner as it extends from the distal end 4b to the proximal end 4a of the coil, as depicted in FIG. 6a, or it can have a uniform filament cross-section, as shown in FIG. 6b. The elongated filament member 4 can also have a coil that tapers outwardly from the distal end 4b to the proximal end 4a, as demonstrated in FIG. 6c or, alternatively, the coil can taper inwardly from the distal end 4b to the proximal end 4a, as depicted in FIG. 6d. Furthermore, as shown in FIG. 6e, the elongated filament member 4 can have a coil that initially tapers outwardly from the distal end 4b towards the proximal end 4a, and then tapers inwardly as it continues towards the proximal end. This design could allow the elongated filament member 4 to better nest within an anatomical space while providing a larger surface area for drug elution. Various arrangements can be formed to accommodate preferred drug elution or delivery rates and amounts.

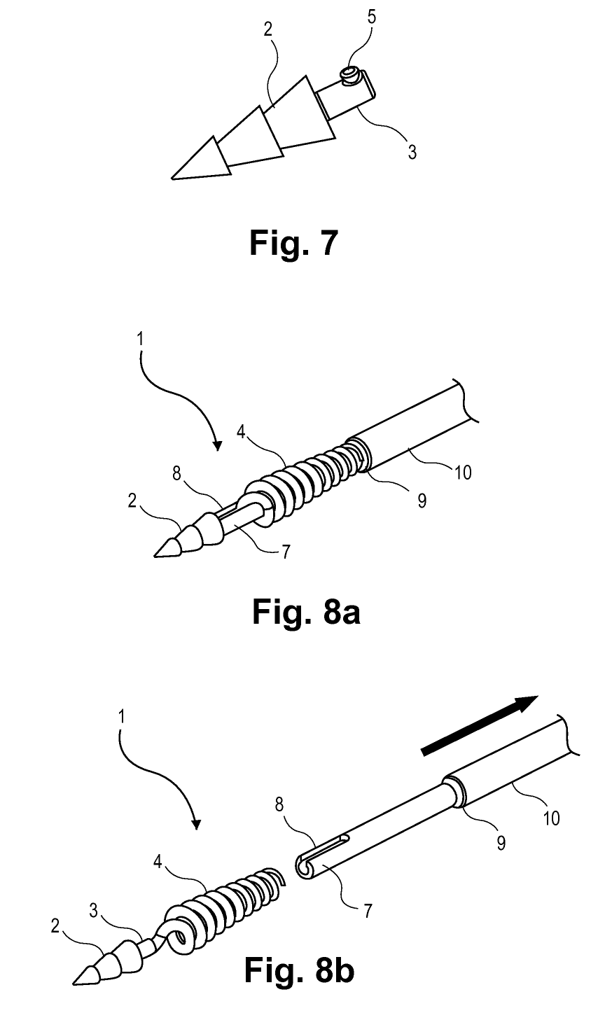

Similar to FIG. 5a, the tip 2 of FIG. 7 could have a boss feature 5 that is part of the interface feature 3 and acts more like a pin-feature, which can be used for controlled retention of the overall tip feature. To further aid in implanting the device without direct visualization, either by an arthroscope or through an open incision, the bioabsorbable filament implant 1 can contain a radiopaque material that would allow the device to be visible using fluoroscopic imaging. The bioabsorbable filament implant 1 at the tip of a delivery tool 13 would be visible under fluoroscopic imaging using a minimally invasive approach either with or without a arthroscopic port, such that the target tissue can be identified and then the bioabsorbable filament implant 1 can be deployed to provide secure fixation to the target tissue. Biocompatible radiopaque medias and salts, known in the art, can be added to the implant material, for example, tantalum, tungsten, barium sulfate, bismuth subcarbonate, as well as many others.

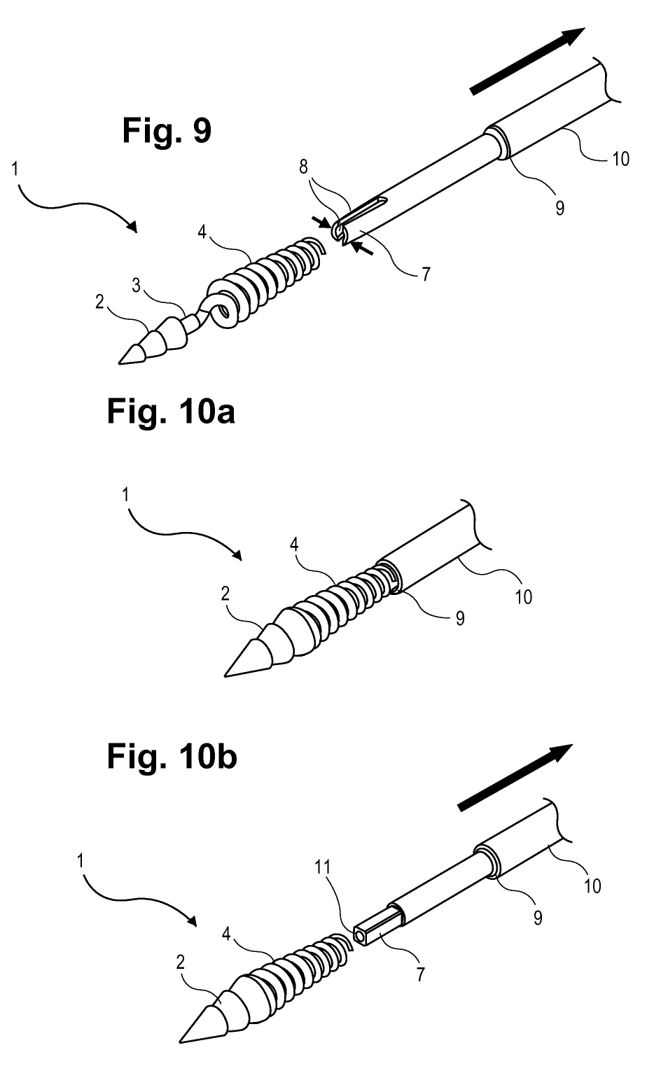

The bioabsorbable filament implant 1, like that in FIG. 1a, can be fitted to a delivery tip 7, depicted in FIG. 8a, by the interface feature 3 (not shown in Figure), where the filament member 4 is accommodated by a slot 8 formed in the delivery tip. The filament member 4 is further supported by a ridge 9 on the delivery shaft body 10. This arrangement permits the tip 2 to be inserted into a suitable substrate, e.g. bone, meniscus, or other aforementioned tissue, and then the delivery tip 7 can be removed from around the interface feature 3, as depicted in FIG. 8b. The inner bore of the delivery tip 7 can have a slight interference fit with the interface feature 3 or the interface feature 3 can have a very small ridge or rib (not shown) that creates an interference fit with internal bore of the delivery tip 7, both of which provide a minimum amount of friction and prevent the bioabsorbable filament implant 1 from releasing prior to implantation. In an alternative embodiment, the delivery tip 7 can have two slots 8, such that the two independent members of the delivery tip 7 can have a slight inward bias, as depicted in FIG. 9, that act as spring elements and provide friction against the interface feature 3, such that it can be controllably released. Additionally, using the delivery tip 7 as currently described, the bioabsorbable filament implant 1 depicted in FIG. 2a can similarly be fitted and deployed.

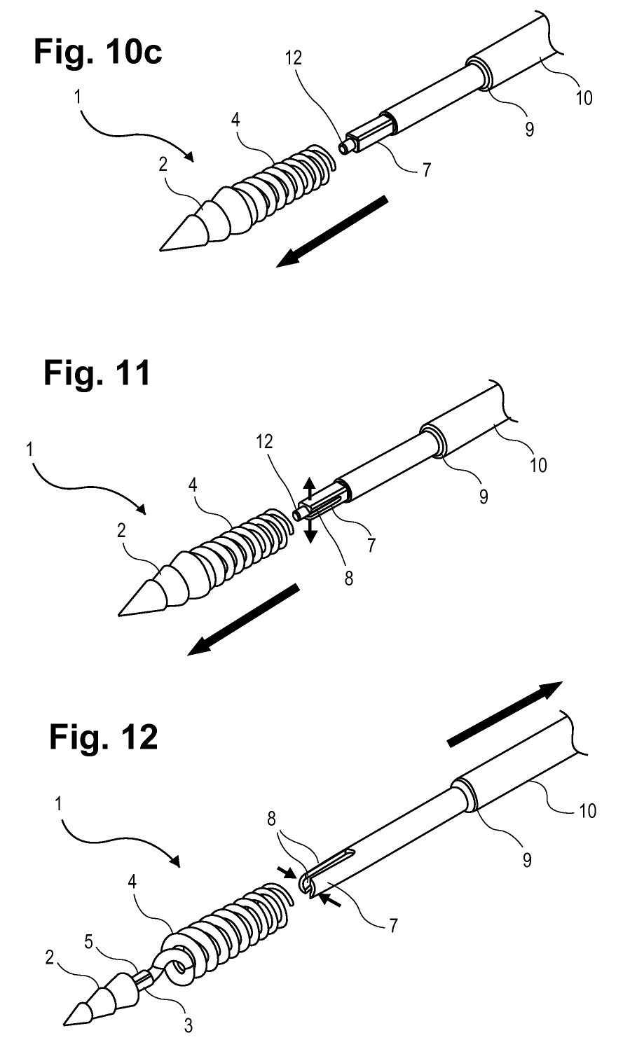

In yet another embodiment of the invention, the bioabsorbable filament implant 1, as depicted in FIG. 3a, can be fitted to a delivery shaft 10, as shown in FIG. 10a. In this embodiment, as shown in FIGS. 10a-10c, the tip 2 is mounted onto a delivery tip 7 that acts as an internal drive and support feature, and the bioabsorbable filament implant 1 is arranged to be inserted into a suitable substrate (not shown) and then the delivery shaft 10 removed to release the implant. The delivery tip 7 can also have a central bore 11, which can accommodate a guidewire or drill guide for over-the-wire applications using a cannulated implant, as described previously. Alternatively, the central bore 11 can contain a pushrod 12 that is slidably controllable and can be used to further drive the bioabsorbable filament implant 1 into tissue and/or to overcome the retention friction between the tip 2 and the delivery tip 7 during implantation. Furthermore, similar to the retention feature in FIG. 9, the delivery tip 7 can also have two slots 8, such that the two independent members of the delivery tip 7 can have a slight outward bias, as depicted in FIG. 11, that act as spring elements and provide friction against the interface feature 3 (not shown), such that it also can be controllably released. Again, the pushrod 12 can provide additional force to release the tip 2 from the delivery tip 7.

To provide additional support to the elongated filament member 4 during manipulation, the boss feature 5 of the bioabsorbable filament implant 1, as depicted in for example FIG. 4, can interface with the slot(s) 8 of the delivery tip 7, as demonstrated in FIG. 12, and prevent rotation of the implant and/or excessive forces against the elongated filament member 4, which could lead to premature fatigue, fracture and/or failure during the implantation process. Additionally, the delivery tips can have a slight inward bias to increase retention of the implant by increasing the sliding friction.

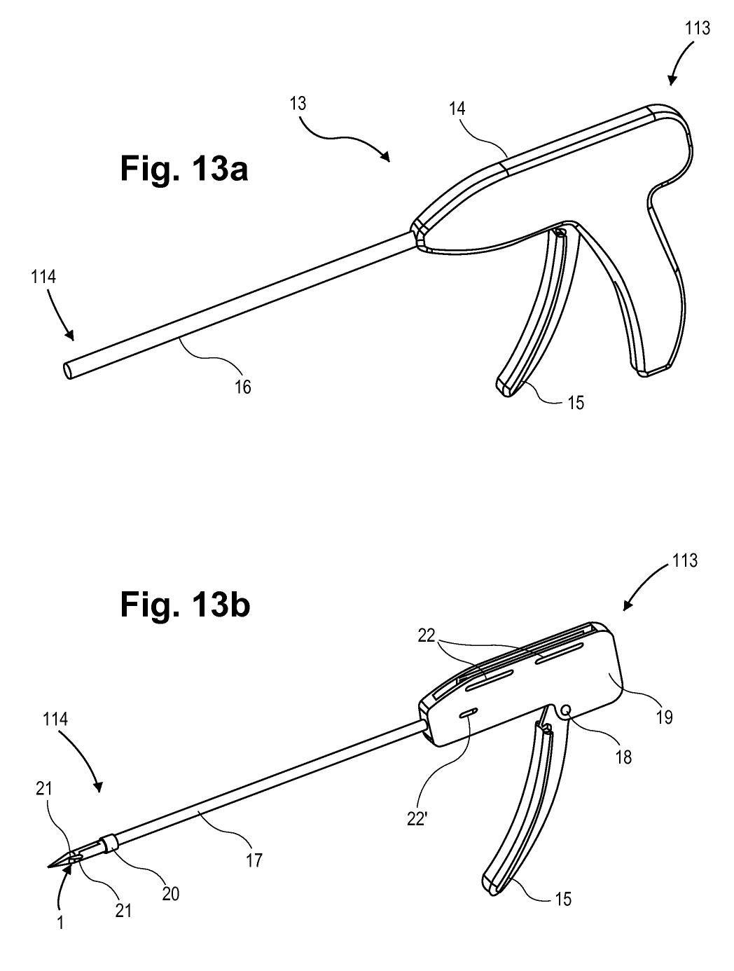

The bioabsorbable filament implant 1 may be delivered from a delivery tool 13, like that shown in FIG. 13a. While delivery tool 13 is specifically described and illustrated herein, any mechanism or device or equivalent suitable for delivery of the implant to the targeted region would be suitable for use. The delivery tool generally includes a proximal portion or end 113 including a handle body 14, a hand-actuated lever 15. The delivery tool further includes an elongated support tube 16 that is fixed to the handle body 14, extends from the handle body to the distal portion or end 114, and can fit within typical arthroscopic ports (not shown). As depicted in the isolated internal components view of FIG. 13b, within the hollow handle body 14 lies a mechanism carriage 19 that further houses the additional components for controlling the actuation and delivery of the bioabsorbable filament implant 1, a pivot pin 18 about which the lever 15 rotates, a push-tube 17 that translates within the support tube 16 while being held concentric by a collar 20, and two pointed penetrating tips 21 that are supported by a flexure, linked to the push-tube 17.

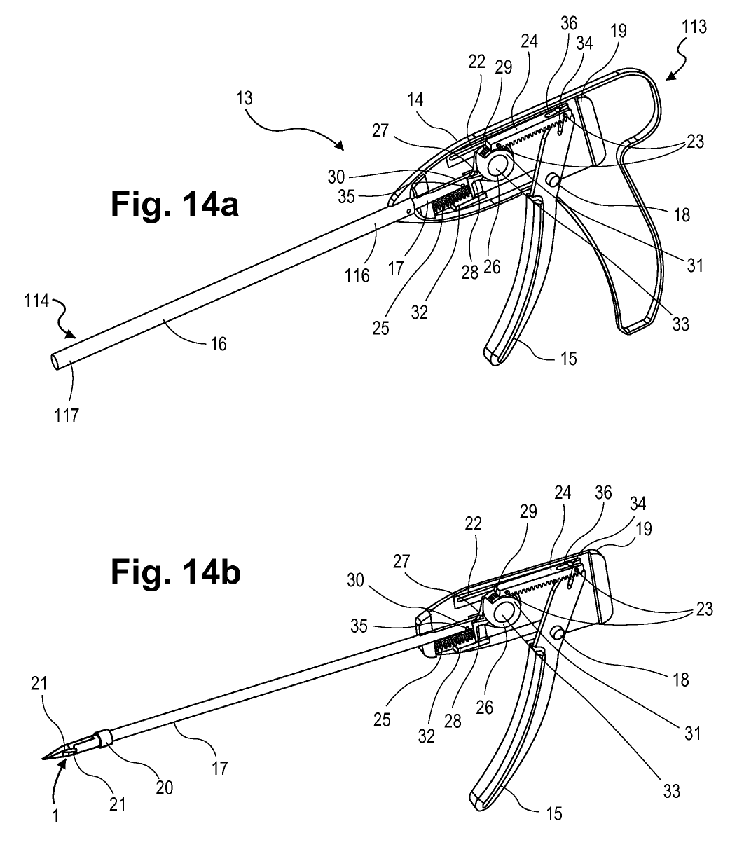

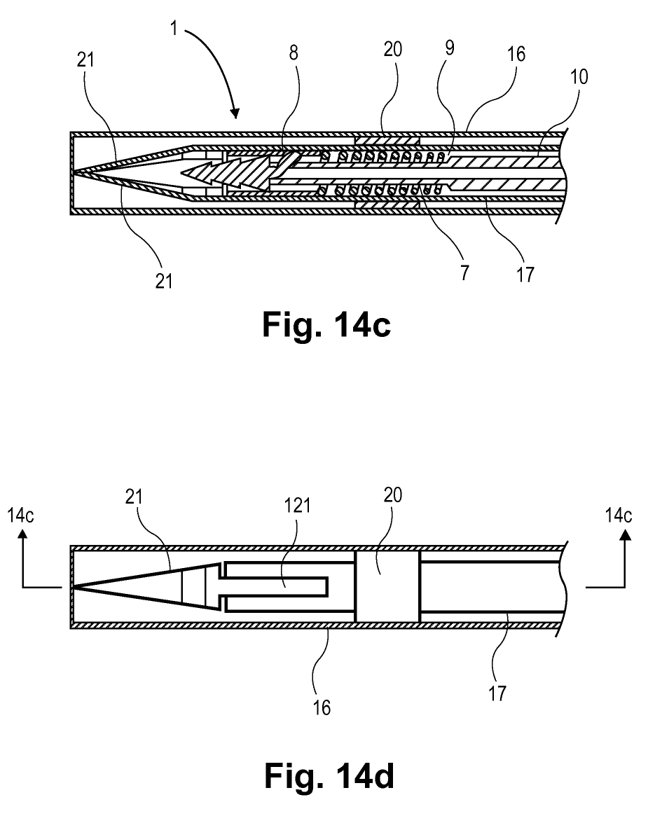

The partial cross-section isometric view of FIG. 14a further depicts the internal mechanism or actuation device housed within the handle body 14 and mechanism carriage 19 that enables the delivery of the bioabsorbable filament implant 1. The lever 15 has a slot 34 that slidably engages with the proximal rack pin 23 which spans the rack slot 36 of the drive rack 24. The lever 15 can also have a return spring (not shown), for example, a torsional, extension or compression spring, that maintains the lever in the extended position, as shown. The free ends of both the distal and proximal rack pins 23 extend beyond the drive rack 24 and slidably engage with the slots 22, also shown in FIG. 13b. The distal-end of the drive rack 24 is fixedly attached with the proximal-end of the pushrod 27, which is in turn directly fixedly attached to the proximal end of the delivery shaft 10 (not shown). The drive rack 24 has gear teeth that freely mesh with the gear 29, which rotates about the hub 33. The gear 29 is mated with a cam 26 by a cross-pin 31 that ensures that the relative timing of the gear 29 and cam 26 does not change. The cam 26 also rotates about the hub 33. The cam 26 is slidably engaged with the cam follower body 28 as it translates in a linear fashion relative to the profile of the cam 26 surface. The cam follower body 28 is also slidably engaged with the support feature 32, which is fixedly attached to the mechanism carriage 19, and provides additional mechanical strength to the cam follower body 28 during actuation. A spring 25 ensures that the cam follower body 28 maintains intimate contact with the profile of the cam 26 surface. In turn, a slotted feature of the cam follower body 28 (not shown) extends into the push-tube 17 and fixedly connects the two components by accommodating two link pins 35. The link pins 35 and slotted feature of the cam follower body 28 (not shown) do not obstruct the central bore of the push-tube 17 in order to allow the push-rod 27 to freely translate. The link pins 35 also extend beyond the push-tube 17 and slidably engage with the push-tube slots 22′, which can be seen in FIG. 13b, for additional support. A push-rod slot 30 in the push-tube 17 and the cam follower body 28 further accommodates the extended travel of the push-rod 27. The support tube 16 is fixedly attached to the handle 14 and houses the distal portion of the tool.

The partial cross-sectional view without the support tube 16 and the handle 14, as shown in FIG. 14b, further depicts the distal portion 114 of the tool which consists of the push-tube 17, the collar 20 and the two pointed penetrating tips 21, which are arranged to cover and constrain the bioabsorbable filament implant 1, prior to delivery. The elongated support tube is formed of an elongate tubular member having a proximal end 116 and a distal end 117 (shown in FIG. 14a). The elongate tubular member has an outer diameter sized to be received within an arthroscopic port. A centralized aperture or bore or passageway extends from the proximal end to the distal end. The elongated support tube is generally adapted to carry the bioabsorbable implant therein.

Generally, a penetration element 21 having a sharpened tip is slidably disposed in the passageway and moveable between a first position in which the tip is recessed within the distal end of the elongate tubular member and a second position in which the tip is at least partially extended from the distal end. The penetration element is adapted to carry the implantable device. The actuation mechanism or device, which is at least partially carried by the elongate member, is capable of moving the penetration element 21 from the first position to the second and for delivering the implantation device from the elongate tubular member into an implanted position in the joint.

The cross-section side-view of the distal portion 114 of the tool, as shown in FIG. 14c, depicts the bioabsorbable filament implant 1 mounted on the delivery tip 7, where the implant is further supported by the ridge 9 on the delivery shaft 10. The push-tube 17 encapsulates the implant and supports two pointed penetrating tips 21 that are independently flexible but come to a point at the most distal portion of the tip.

The pointed penetrating tips 21 can be manufactured from metal, for example from the push-tube material itself, by conventional machining, computer numerical control (CNC) machining, electric discharge machining (EDM), grinding and/or laser-cutting and then forming by conventional sheet metal techniques, hydro-forming, and/or die-forming. Additionally, the pointed penetrating tips 21 can be formed, die-cut and/or machined separately and then fixed to the push-tube 17 by fasteners, pins, welds, adhesive, or any other method known in the art. One exemplary embodiment is shown in FIG. 14d, in which pointed penetrating tips 21 have an arm 121 or pin or more than one arm or pin that engages or is received by push tube 17 at its distal end. The arm(s) 121 may be received within a corresponding shaped recess or aperture in the push tube or may be attached or adhered to the a surface of the push tube. Furthermore, the push-tube 17 and the pointed penetrating tips 21 can be made from a polymer, e.g. acetyl, polyetheretherketone (PEEK), polyolephin, polyethylene, or any other polymer known in the art. Additionally, the push-tube 17 could be made from a polymer material and the pointed penetrating tips 21 can be made from metal and connected using methods described earlier, as known in the art. Additionally, while “pointed” penetrating tips 21 are specifically described, alternative geometries would not depart from the overall scope of the present invention. Likewise, while two tips 21 are specifically described, more than two tips are also contemplated.

The collar 20 is fixedly attached to the push-tube 17 and is capable of freely translating within the support tube 16, as the tool mechanism is actuated. The collar 20 could be made from a polymer to ensure smooth translation within the support tube 16 with little or no lubrication. Additionally, the collar 20 can act as a joint or union with which to connect the push-tube 17 to the distal end which contains the pointed penetrating tips 21, by means of a press-fit or threads or adhesives or set-screws. To enable the push-tube 17 to be assembled from independently manufactured components.

The top-view of the distal portion 114 of the tool, as depicted in FIG. 14d, shows the flexure-like feature of the pointed penetrating tip 21 as it extends from the push-tube 17, which is all housed in the support tube 16.

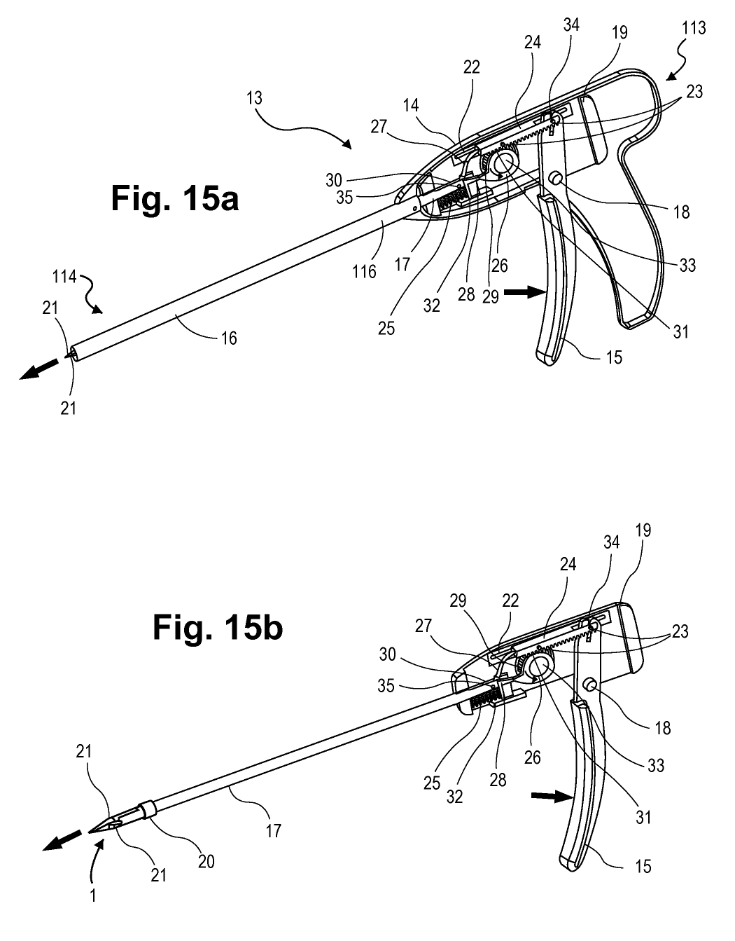

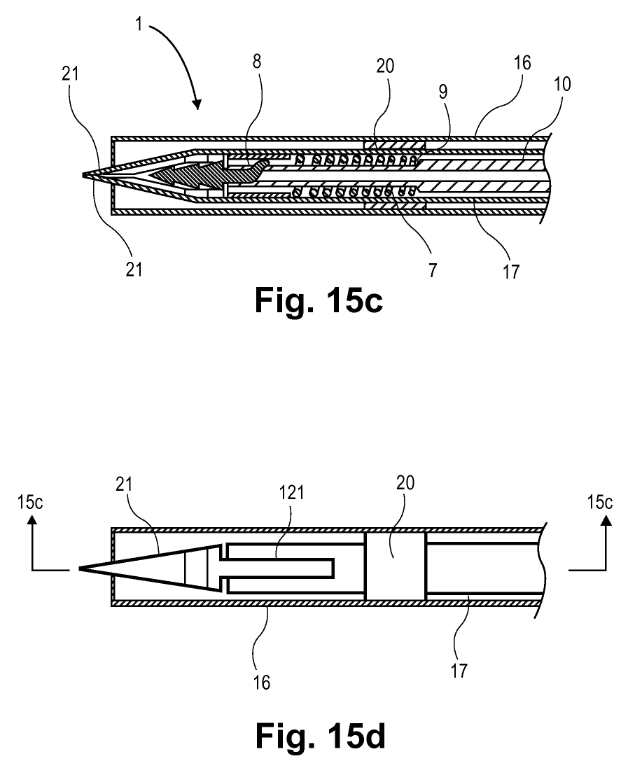

In operation of the delivery device, as the lever 15 is actuated by the user's hand, as indicated by the arrow in FIG. 15a, the lever 15 rotates about the pivot pin 18 to act on the proximal rack pin 23 that is slidably constrained within the slot 34. In turn, the drive rack 24 advances distally and rotates the gear 29, which in turn rotates the cam 26. The cam follower body 28 follows the cam 26 surface and advances distally, where it reaches its maximal position, as shown in FIG. 15a. The push-tube 17 also advances distally, based on the translation of the cam follower body 28 to its maximum position, as shown in FIG. 15b. The two pointed penetrating tips 21 extend beyond the support tube 16, shown in FIG. 15c, a distance suitable to initially puncture the target tissue, bone, substrate or intended target material and therefore facilitate the entry of the bioabsorbable filament implant 1. The pushrod 27, which is also advanced distally by the drive rack 24, translates the delivery shaft 10 which in turn advances the bioabsorbable filament implant 1 distally within the push tube 17, as indicated in FIGS. 15c-15d.

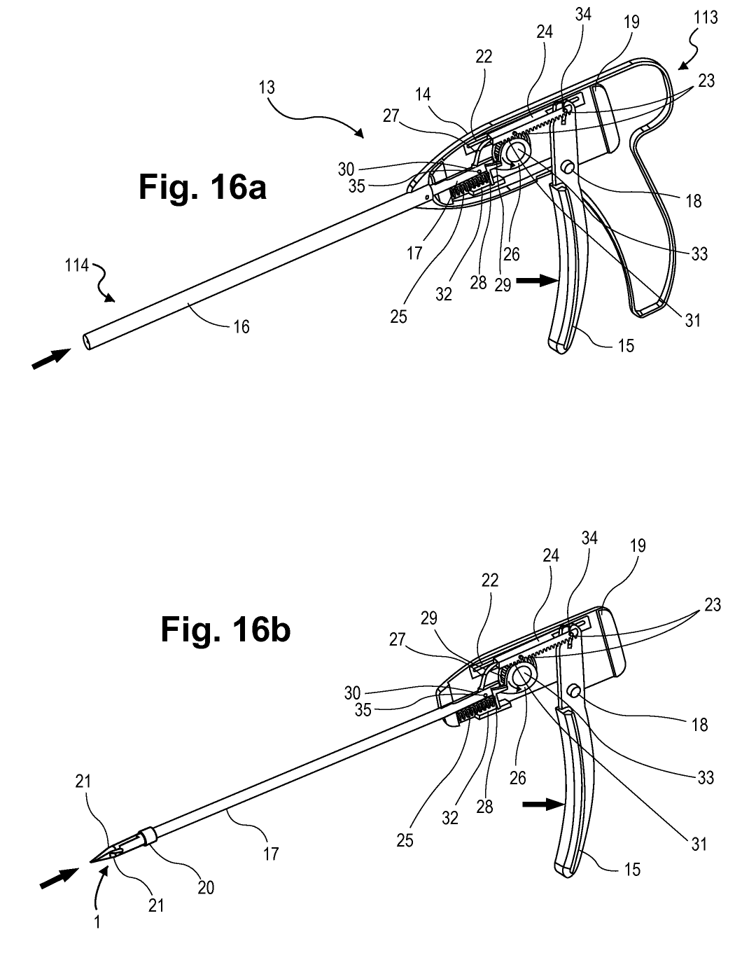

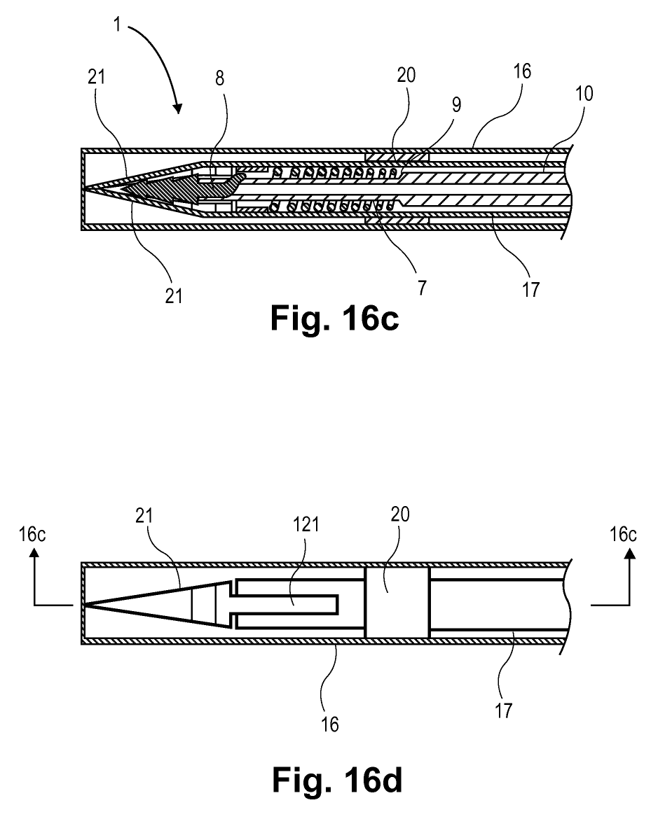

As the lever 15 further rotates about the pivot pin 18, the drive rack 24 continues to advance distally and further rotates the gear 29, which in turn continues to rotate the cam 26. The cam follower body 28 continues to follow the cam 26 surface under the spring tension provided by the spring 25 and retracts proximally as it just passes beyond the maximum height of the cam 26 lobe, as shown in FIG. 16a. Correspondingly, the push-tube 17 retracts proximally, as depicted in FIG. 16b, based on the retracted position of the cam follower body 28. The two pointed penetrating tips 21 also retract proximally just within the support tube 16, as depicted in FIG. 16c. The pushrod 27 advances distally incrementally, which further translates the delivery shaft 10 distally that in turn advances the bioabsorbable filament implant 1 distally within the push tube 17, as indicated in FIGS. 16c-16d. In a preferred embodiment, the cam mechanism design and timing ensure that the position of the bioabsorbable filament implant 1 does not deform the two pointed penetrating tips 21 while transitioning into their retracted state, as also shown in FIGS. 16c-16d, in order to prevent the two pointed penetrating tips 21 from separating while still within the tissue, which could cause tissue tearing or interfere with the implantation of the device.

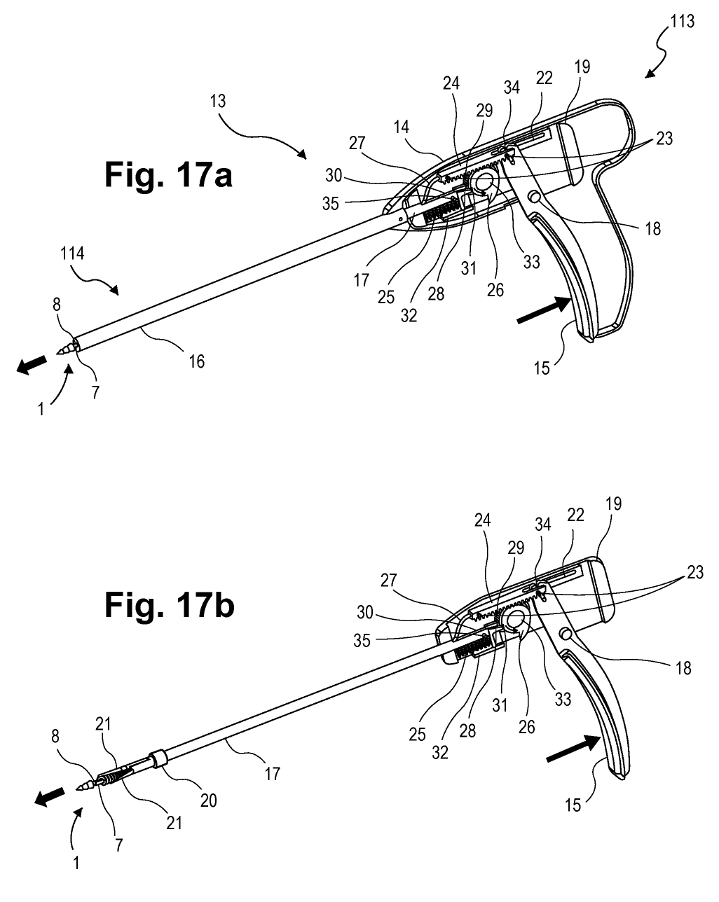

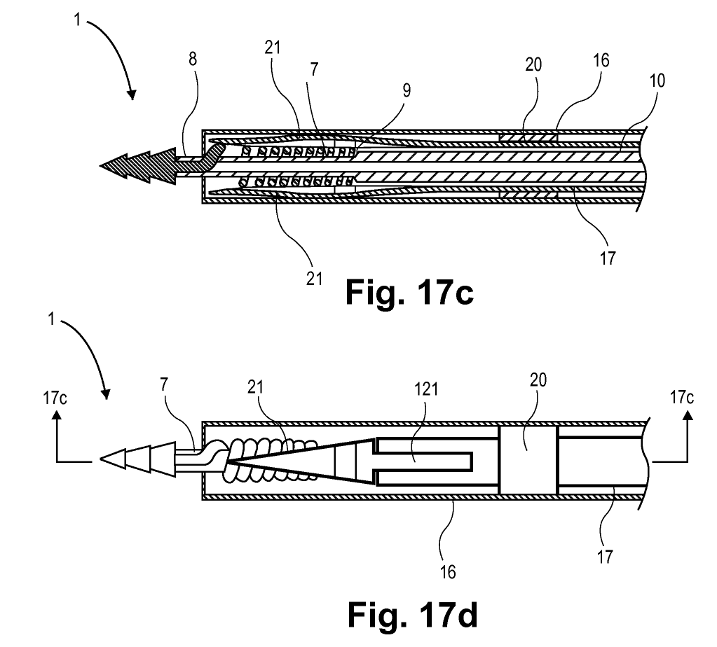

Further advancement of the lever 15, as shown in FIG. 17a, advances the drive rack 24 even more distally which in turn rotates the gear 29 and the cam 26. Both the cam follower body 28 and the push-tube 17 remain in the retracted position, while under the spring tension provided by the spring 25. The pushrod 27 advances, which translates the delivery shaft 10 distally that advances the bioabsorbable filament implant 1 distally. Preferably, the two pointed penetrating tips 21 deflect by means of their flexure like feature, shown in FIG. 17b, by utilizing the tip 2 of the bioabsorbable filament implant 1 as a wedge while driven distally, until the tip 2 is entirely exposed relative to the support tube 16, as shown in FIGS. 17c-17d. Furthermore, the delivery tip 7 extends beyond the support tube 16 and serves to further drive the bioabsorbable filament implant 1 into the target substrate (not shown), as depicted in FIGS. 17c-17d.

The push-tube 17 can be lined with an additional material (not shown) in order to provide an even more lubricious surface between the bioabsorbable filament implant 1 and the two pointed penetrating tips 21 and to also protect the typically fragile and brittle bioabsorbable materials, known in the art, from cuts, gouges, scoring, abrasion or other surface defects caused by the relative motion of the implant and the pointed penetrating tips 21. Alternatively, the additional material (not shown) can just be isolated to the inner surface of the two pointed penetrating tips 21. The additional material can be a coating, a layer of polymer attached, fused or glued to the inner surface of the pointed penetrating tips 21 or can merely be another tubular component that fits within the push-tube 17 with features that match the shape of the pointed penetrating tips 21 and acts merely as a liner.

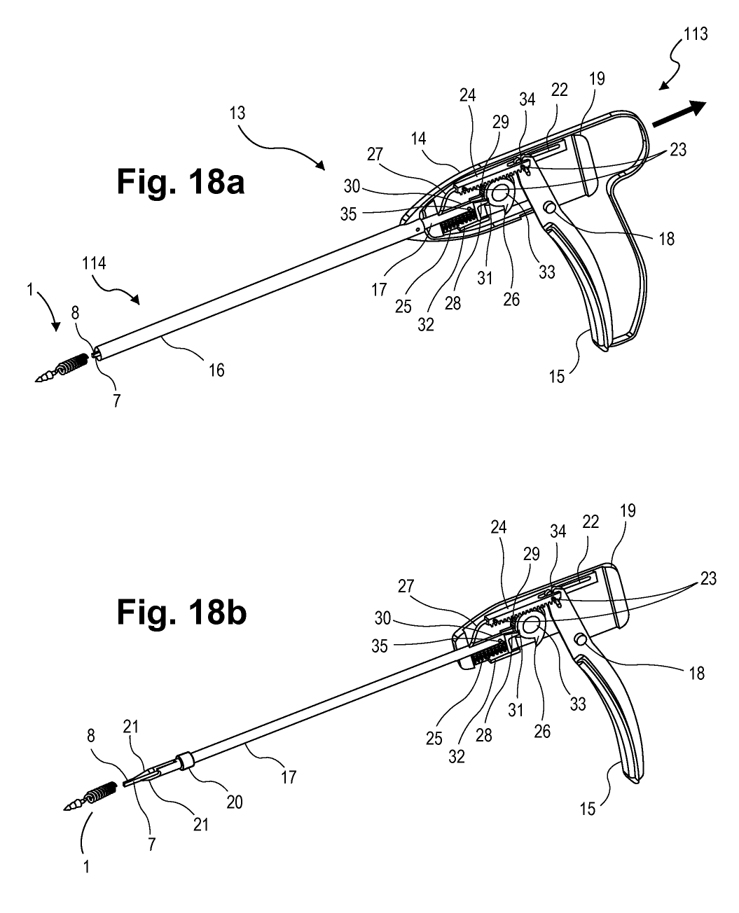

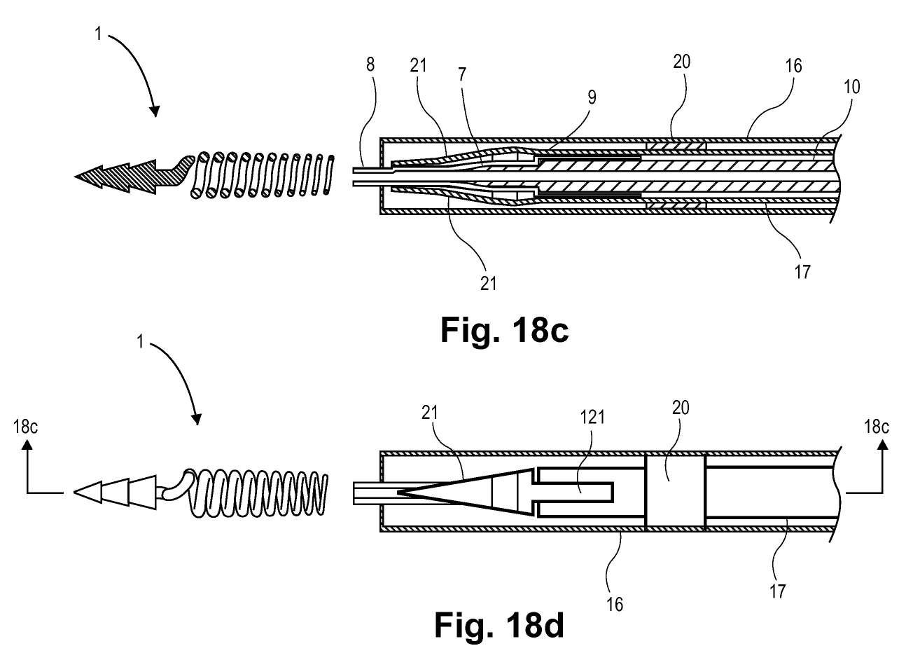

With the bioabsorbable filament implant 1 fixed within a target substrate (not shown), the delivery tool 13 can be retracted proximally to release the implant from the delivery tip 7, as shown in FIGS. 18a-18d. Additionally, the implant can also be further “ejected” from the delivery tip 7 with the previously described pushrod 12 arrangement. In one embodiment, when the lever 15 is released, the cam follower body 28 catches the lobe of the cam 26 and prevents the mechanism from “resetting” to its original starting position, as provided by the handle return spring (not shown). This features serves as a safety mechanism or “lockout” and prevents the pointed penetrating tips 21 from being exposed after the tool has delivered the implant and the force against the lever 15 has been removed.

As discussed, in order to provide a variety of medicaments to properly treat a particular anatomical site, multiple bioabsorbable filament implants 1 that contain different medications and/or with different doses can be introduced into the target tissue or joint. To this end, the delivery tool 13 could be modified for implanting multiple bioabsorbable filament implants 1 by having an exchangeable front end with a specific bioabsorbable filament implant 1 that resets the mechanism within the handle body 14 for another deployment. Alternatively, the delivery tool 13 could be modified to accommodate multiple bioabsorbable filament implants 1 from an internal cartridge (not shown) or cassette (not shown), similar to a surgical stapler, in which case the user would merely actuate the handle cycle repeatedly to deliver multiple devices into a target region.

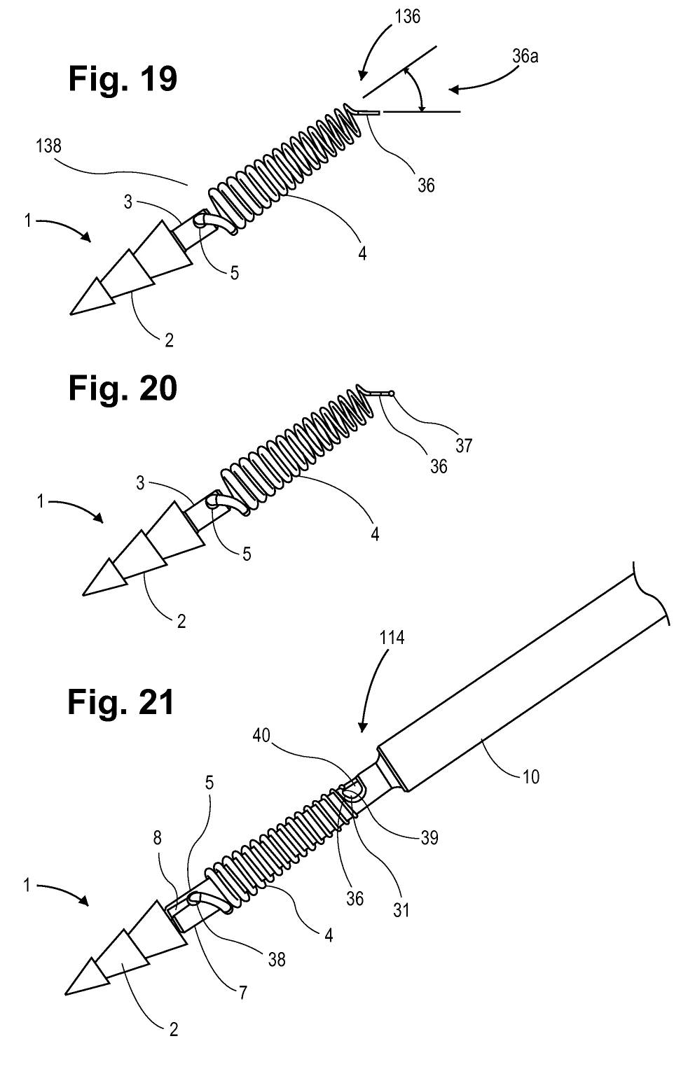

In another embodiment of the invention, the bioabsorbable filament implant 1, like that depicted in FIG. 1a, could have a tail 36 feature on the proximal end 136 of the elongated filament member 4 that deviates from the coil pattern and angles towards the Distal-Proximal axis, formed by the central axis of the coiled filament shown in FIG. 19 extending between the proximal end 136 and distal end 138, with a tail angle 36a range of approximately 0 degrees to 90 degrees, or more specifically approximately 30-60 degrees, or even more narrowly 40-50 degrees. This tail 36 feature can be used to capture and constrain the proximal portion of bioabsorbable filament implant 1 for additional control and/or capture of the overall implant body.

In another embodiment, a tail catch 37, as shown in FIG. 20, could also be incorporated in the filament implant 1 that would provide another means to capture and constrain the tail 36, by either serving as the capture feature specifically or by acting as a stop and preventing the tail 36 from slipping through a compression mechanism (not shown) that clamps on the outside or periphery of the tail 36. In a preferred embodiment, the tail catch 37 is arranged to hold the filament 1 in place and constrain the filament from falling off or separating from the end of the tool. The tail catch 37 may then optionally be removed for insertion. The distal end 114 or tip of the tool, as demonstrated in FIG. 21, provides an example of the compression mechanism. The delivery shaft 10 and delivery tip 7 resemble the distal tip of the tool featured in the example FIG. 8b, however, the boss feature 5 of the bioabsorbable filament implant 1 is captured in a notch 38 on the proximal corner edge of the slot 8, as depicted in the isometric view of FIG. 20. The tail 36 is clamped between a slidable proximal jaw 40 that translates within the delivery shaft 10 and a fixed distal jaw 41 that is accessible through the window 39 in the delivery tip 7, as demonstrated in FIG. 21. Additionally, torsional tension applied to the proximal end of the filament member 4 to constrict or reduce the coil diameter helps to further engage the boss feature 5 within the notch 38. By locking the tail between the clamping mechanism of the fixed distal jaw 41 and the slidable proximal jaw 40 while in this torsionally constricted state enables the bioabsorbable filament implant 1 to be fully captured distally within the notch 38 and proximally with the clamping mechanism, in order to better capture the implant.

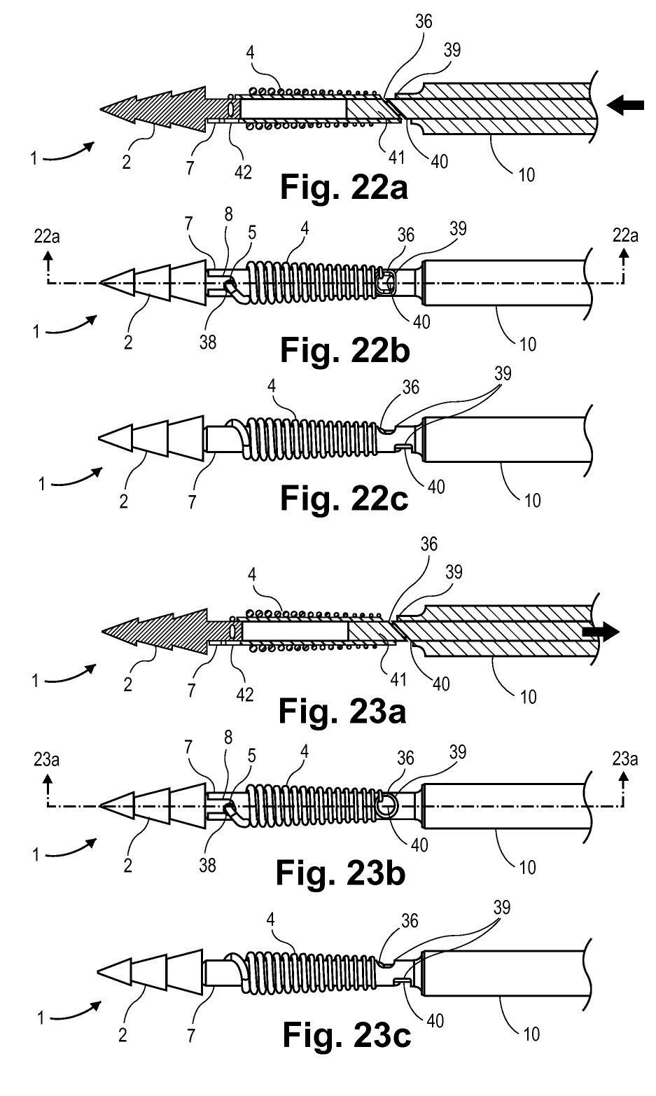

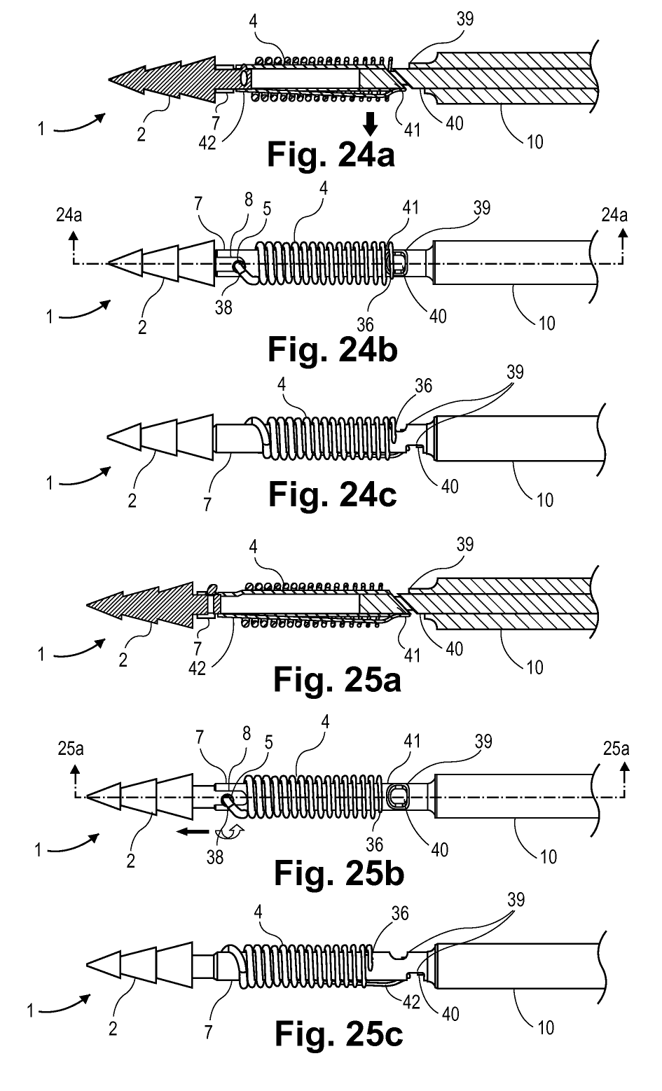

The cross-sectional side view depicted in FIG. 22a further illustrates a slidable proximal jaw 40 that applies a force, as shown by the arrow, which may be used to clamp the tail 36 against fixed distal jaw 41, which is accessible by the window 39 that joins with a matching window on the contra-lateral surface of the delivery tip 7. A release spring 42 is fitted within a groove of the delivery tip 7 and is fixed at its distal end. The release spring 42 nests within the filament member 4. The surfaces of the fixed distal jaw 41 and the slidable proximal jaw 40 can be made from metal or polymer and can have a polymeric and/or elastomeric surface (not shown) that provides a conforming and not-damaging clamping surface for the tail 36 of the bioabsorbable filament implant 1. The top and side view of the distal tip of the tool, as shown in FIGS. 22b-22c, further illustrate the clamping mechanism, where the contra-lateral window 39 can be seen in FIG. 22c. As the slidable proximal jaw 40 is retracted proximally to release the tail 36, which is shown in FIGS. 23a-23c, the release spring 42 is allowed to deflect, as represented by the arrow, and helps to withdraw the tail 36 from the window 39 and to help expand the coiled configuration of the filament member 4, depicted in FIGS. 24a-24c, which may have taken a set while in its constricted state. Additionally, the release spring 42 facilitates the expansion of a more compliant or deformable filament member 4 which does not have the inherent springiness or expandability as compared to a stiffer, more resilient material. The bioabsorbable filament implant 1 rotates out of the notch 38, as depicted by the arrow in FIG. 25b, and can now freely slide off the delivery tip 7. The bioabsorbable filament implant 1 can now be released from the delivery tip 7, as shown in FIGS. 25a-25c. The shape of the notch 38 may be provided with a shallow or deep groove in order to dictate the retention force while the filament member 4 is in a constricted state. The notch 38 can have a sharp or soft corner leading out into the groove 8 and this can determine the ease with which the implant slides off the delivery tip 7, once the tail 36 is released.

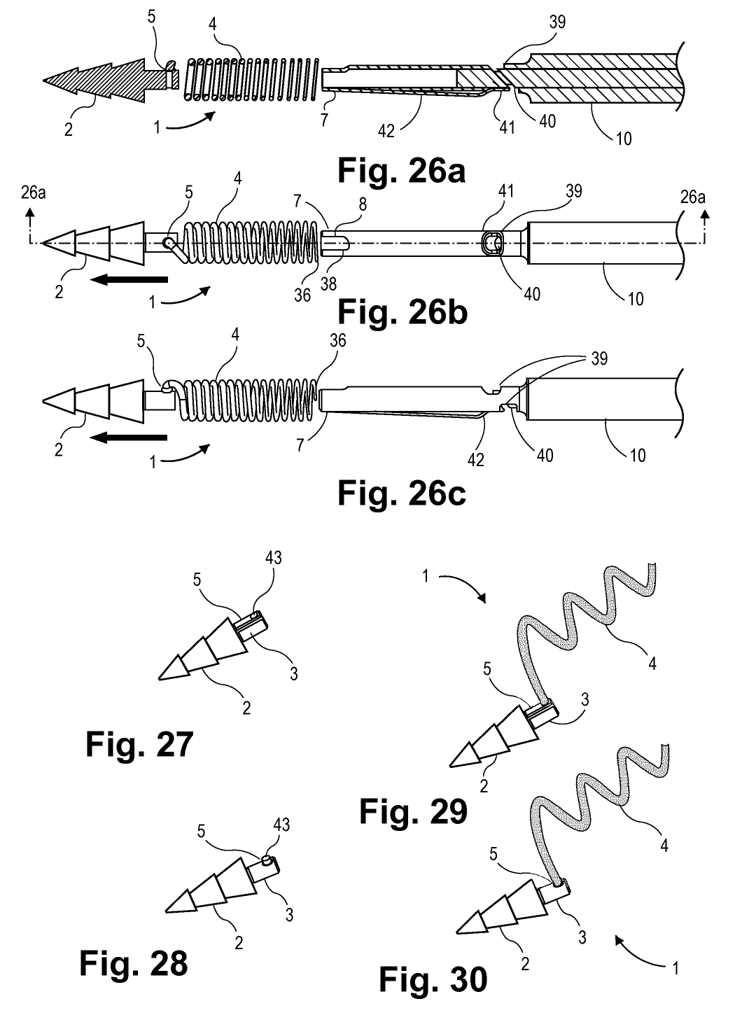

The deployed bioabsorbable filament implant 1 can be deposited into the target substrate (not shown), as depicted in FIGS. 26a-26c, where the arrow merely indicates the relative motion between the implant and the delivery tip 7, which could similarly be achieved by retracting the delivery tip 7 relative to the implant. Additional embodiments of the tips 2 with cross-holes 43 employing elongated or pin-like boss features 5 are depicted in FIG. 27 and FIG. 28, respectively. The cross holes 43 can accommodate filament elements 4 of a different material and/or with different mechanical properties, as suggested by bioabsorbable filament implant 1 shown in FIG. 29 and FIG. 30. The filament element 4 can be secured within the cross-hole 43 by a press fit or with adhesive, ultrasonic welding, uv-cure epoxy, or any other method know in the art. Additionally, the filament element 4 can be over-molded to create the tip 2, interface feature 3, boss feature 5, and cross-hole 43 to create a unibody bioabsorbable filament implant 1, like those depicted in FIG. 29 and FIG. 30.

In a preferred embodiment, the filament is implanted into the joint, preferably not between the articular surfaces, and dissolves after a specific amount of time due to its solubility in the joint fluid. The implant device can be delivered, for example, by an arthroscopic grasper and placed into the joint through an arthroscopic portal. Alternatively, the filament can be placed into a joint space by a purposely designed delivery tool that allows for the tip of the device to more easily fit within an arthroscopic portal and provides more control with regards to the placement and delivery of the implant device.

Careful placement of the filament ensures that it does not interfere with normal joint function, especially during rehabilitative exercises and treatment. In the shoulder joint, for example, the filament device can be placed in the inferior gutter. In the knee joint, for example, the filament can be placed in the supra-patellar pouch or in the medial or lateral gutters.

In order to provide a variety of medicaments to properly treat a particular anatomical site, multiple bioabsorbable filament implants 1 that contain different medications and/or with different doses can be introduced into the target tissue or joint.

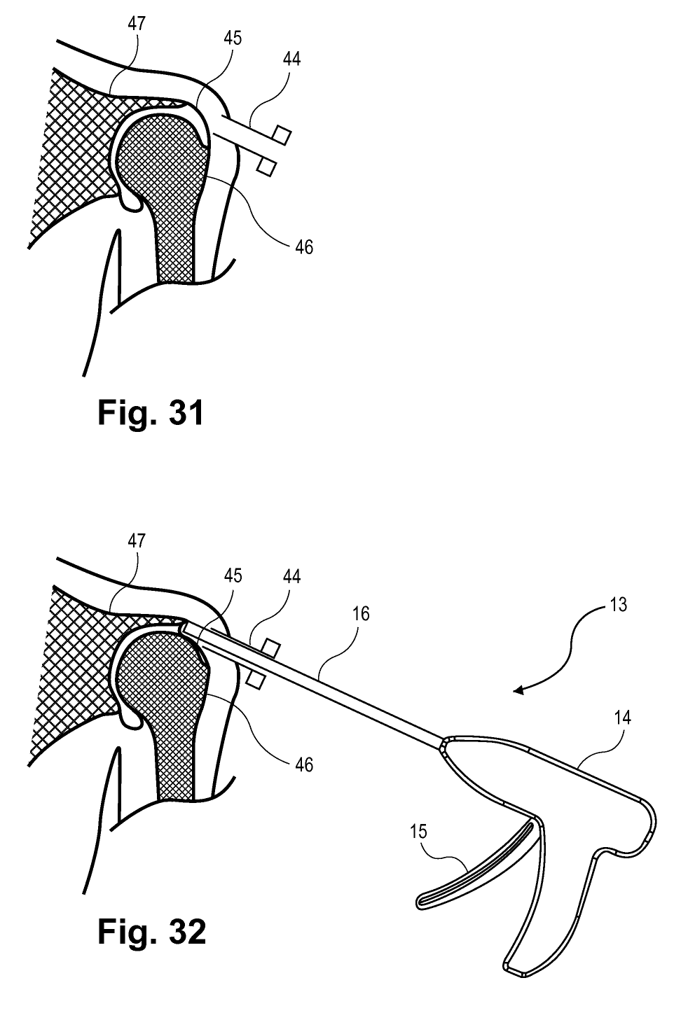

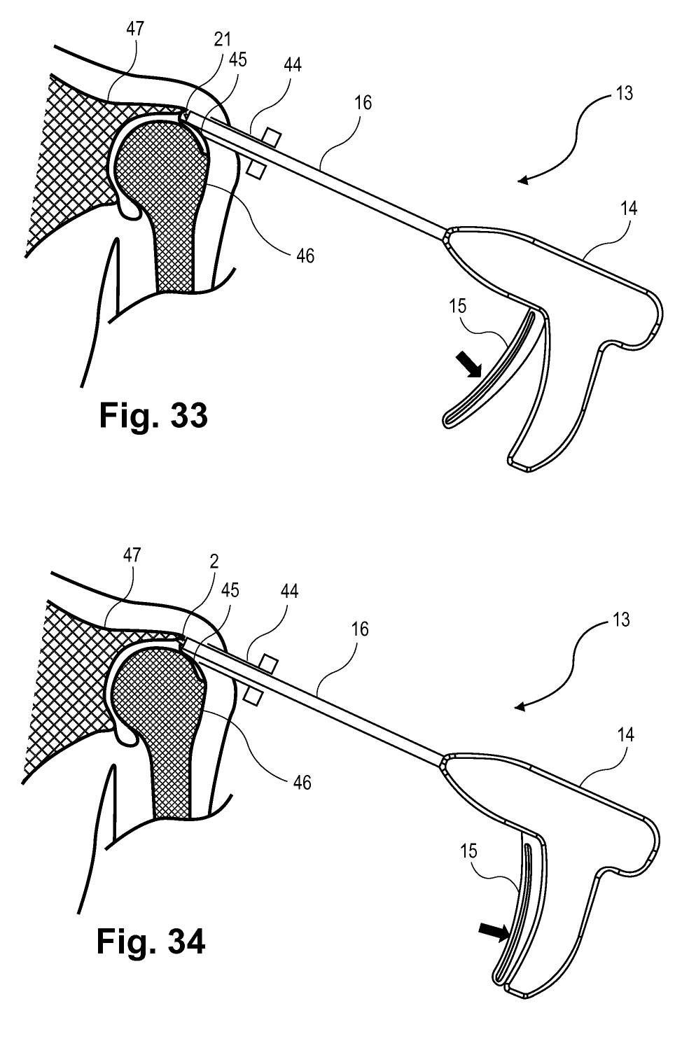

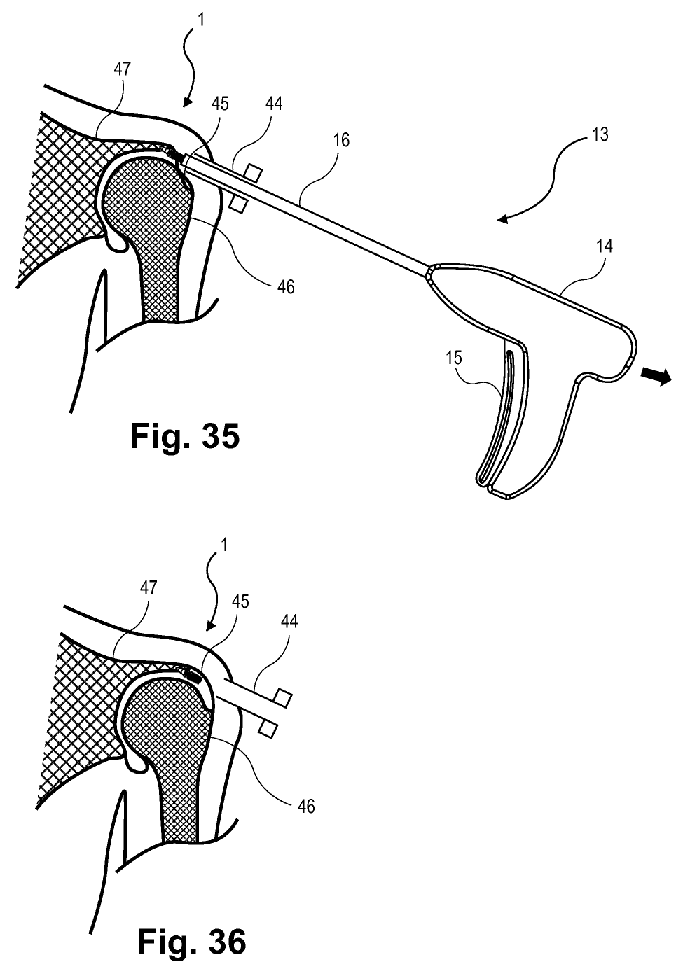

During arthroscopic shoulder surgery, an arthroscopic port 44 is placed, using known surgical techniques, in order to provide sealed access to the capsule 45 which contains the articulating joint between the head of the humerus 46 and the glenoid of the scapula 47, as shown in FIG. 31. The support tube 16 of the delivery tool 13 is inserted through the port 44 and firmly pressed against (or approximated to) the capsule 45 and the scapula 47, like that shown in FIG. 32. Clinically, the support tube 16 would be in intimate contact to the target tissue, bone, substrate or intended target material and when the two pointed penetrating tips 21 retracted, the bioabsorbable filament implant would be further driven distally into the target tissue where the tip 2 would engage and anchor the bioabsorbable implant 1. To this end, the handle 15 is actuated by the user, as shown by the arrow in FIG. 33, to advance the delivery tool 13 mechanism described previously, whereby the two pointed penetrating tips 21 puncture and penetrate the tissue/bone in order to provide an initial hole with which to insert the implant. While maintaining apposition of the support tube 16 with the tissue/bone surface, the handle 15 is completely actuated by the user to completely drive the tip 2 into the scapula 45, for example, as shown in FIG. 34. The delivery tool 13 is then retracted by the user, as shown by the arrow, and the bioabsorbable filament implant 1 remains fixed to the bone, as depicted in FIG. 35. The delivery tool 13 is removed from arthroscopic port 44 and the capsule 45 is repaired, if necessary, using surgical techniques known in the art. The bioabsorbable filament implant 1, as shown in FIG. 36, is located in an area that will not interfere with the normal shoulder function and not be impinged between the articular surfaces. The arthroscopic port 44 can be removed and the skin repaired using techniques known in the art to complete the surgical procedure.

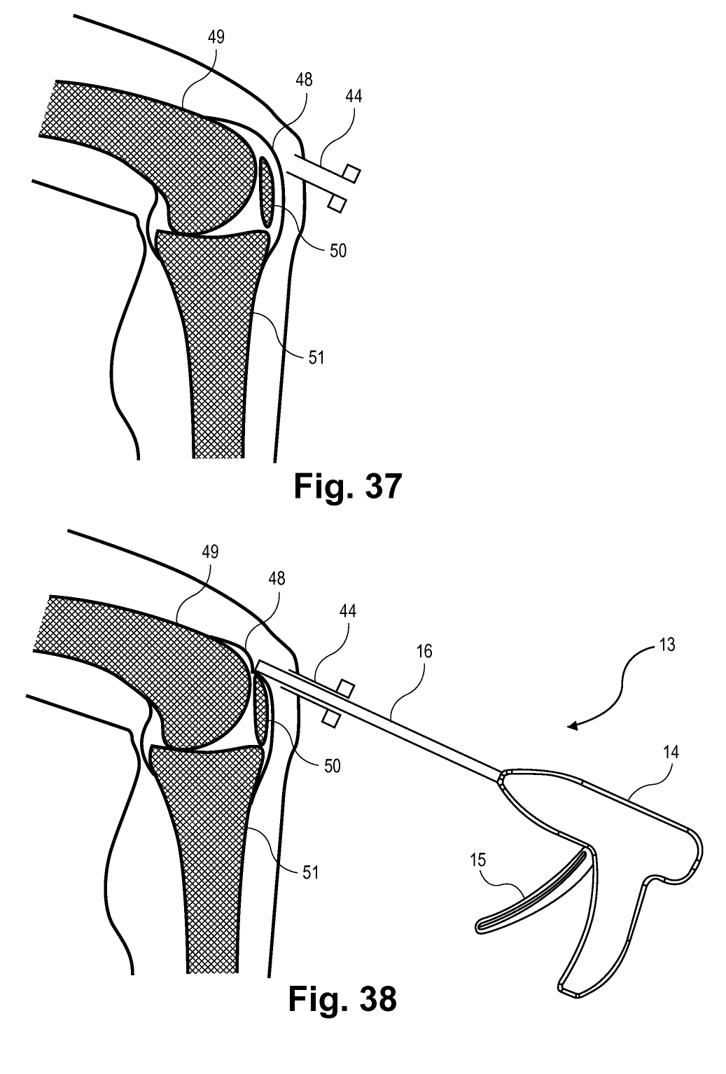

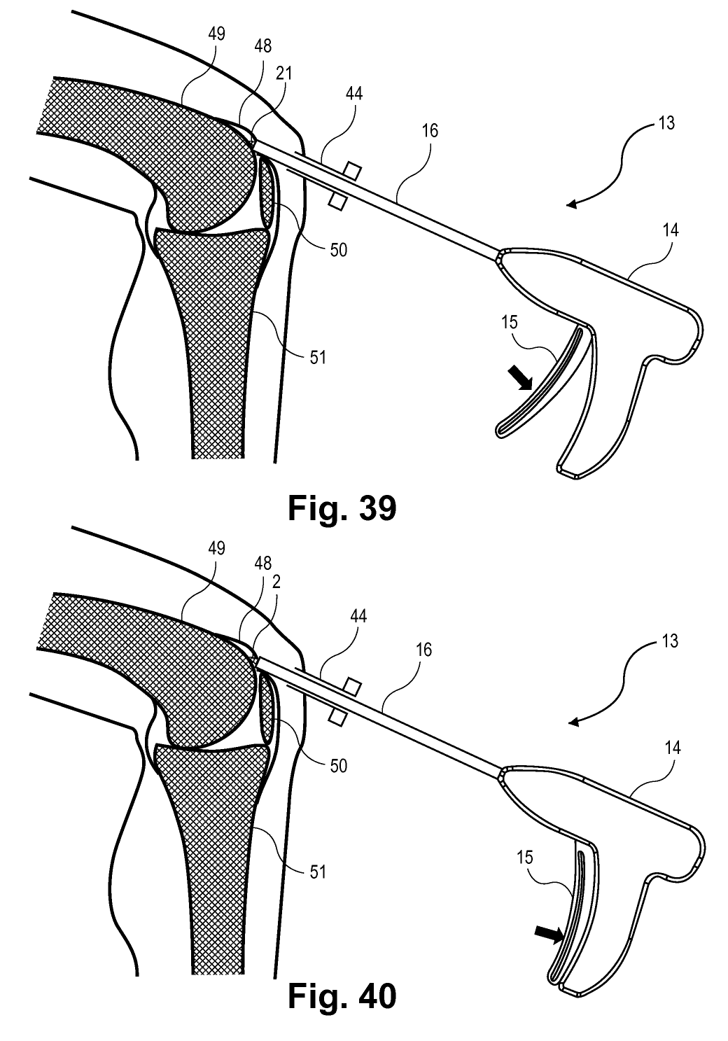

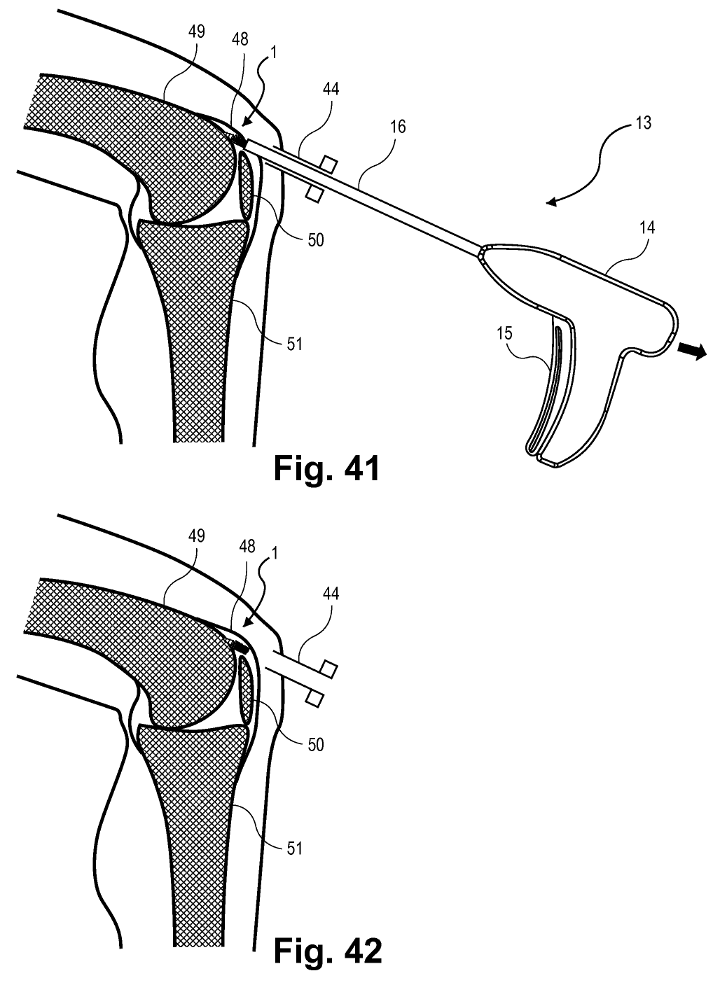

A similar procedure can also be performed on the knee joint, where an arthroscopic port 44 is placed near the patella 49, in order to access the knee capsule 48 that encapsulates the condyles of the femur 48 and the tibial plateau of the tibia 50, as shown in FIG. 37. The lateral and medial condyles are interconnected by a “channel” called the patellar groove (not shown) that helps guide the patella 50 and the patellar tendon (not shown) during extension and flexion of the knee joint. The support tube 16 of the delivery tool 13 is inserted into the arthroscopic port 44 and located against the knee capsule 45 on either side of the patellar tendon (not shown), as depicted in FIG. 38. As in the shoulder example, the handle 15 of the delivery tool 13 is actuated, as shown by the arrow, to deploy the pointed penetrating tips 21 that puncture the tissue/bone in order to provide an initial hole with which to insert the implant, as demonstrated in FIG. 39. By fully actuating the handle 15, as shown in FIG. 40, the tip 2 is completely imbedded in the patellar groove, for example, of the femur 49 to secure the implant. The delivery tool 13 is retracted, as shown in FIG. 41, and the bioabsorbable filament implant 1 remains imbedded in the femur 49. As in the shoulder, the delivery tool 13 is removed from arthroscopic port 44 and the knee capsule 48 is repaired, if necessary, using surgical techniques known in the art. The bioabsorbable filament implant 1, as shown in FIG. 42, is located in an area that will not interfere with the normal knee function. The arthroscopic port 44 can be removed and the skin repaired using techniques known in the art to complete the surgical procedure.

While a shoulder and a knee are specifically described and illustrated, the methods described herein may be applied to alternative locations or joints of the mammalian body without departing from the overall scope of the present invention.

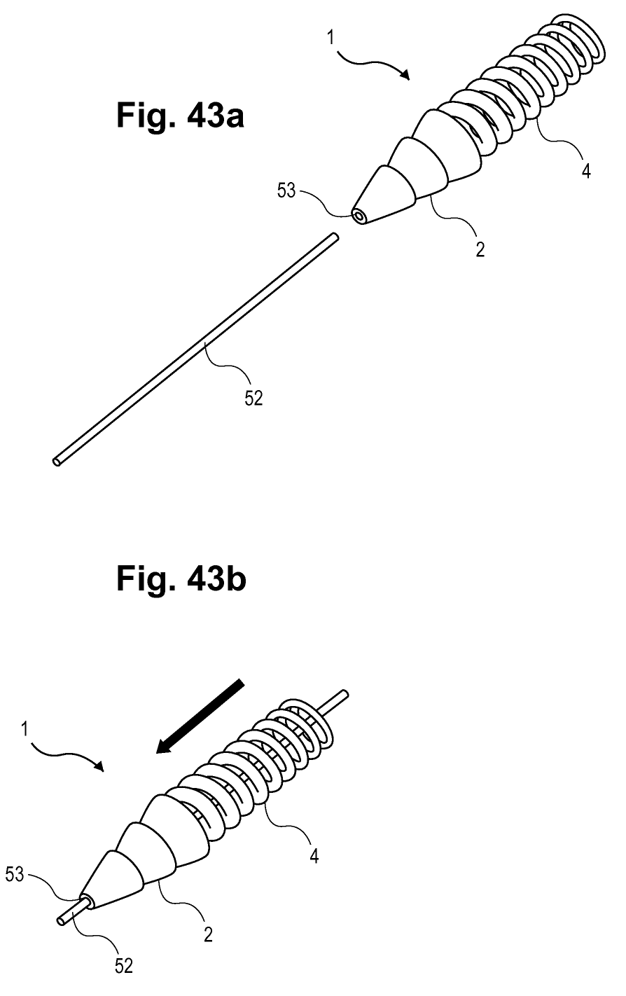

As an alternative implantation method, a drill or punch could be used to make a small hole (or defect) in the target tissue (e.g. bone, cartilage, etc) through an arthroscopic port or directly through the skin, without the use of a port. A drill-guide, which is a wire or rod used to help direct cannulated drills to their target tissue, or guidewire, which is an elongated thin shaft typically used as a guide rail for surgical devices during minimally invasive surgery, could be place in the newly created hole. Additionally, a simple stainless steel rod or wire could also be used. Furthermore, any other biocompatible material could also be used, including metals and polymers. Using an alternative embodiment of the invention, the bioabsorbable filament implant 1, like the implant shown in FIGS. 3a-3c, could be centrally cannulated with a lumen 53 along its long axis to accommodate a guide rail 52, which can be a drill guide, guidewire, wire, or similar type device, as depicted in FIG. 43a. The bioabsorbable filament implant 1 with a lumen 53 can freely slide along the guide rail 52, as illustrated in FIG. 43b. Similar to the other embodiments previously disclosed, the bioabsorbable filament implant 1 with a central lumen 53 can be fitted to a delivery tip 7 (not shown) or incorporated into a delivery tool 13 (not shown) that can also accommodate a guide rail 52.

In yet another embodiment of the invention, the tip 2 of the bioabsorbable filament implant 1 could have threads, as show in FIG. 44a, such that the implant can be directly inserted into tissue by rotating the tip 2 to engage the threads within the tissue. The bioabsorbable filament implant 1 could be mounted on a delivery tip 7 which is then fitted to a ribbed handle 54, as depicted in FIG. 44b, for manual implantation of the implant. Additionally, the bioabsorbable filament implant 1 in FIG. 44a could be incorporated into a delivery tool 13 (not shown), like that previously described, which pierces the tissue and then a rotating mechanism can be incorporated to drive the threaded tip 2 into the tissue.

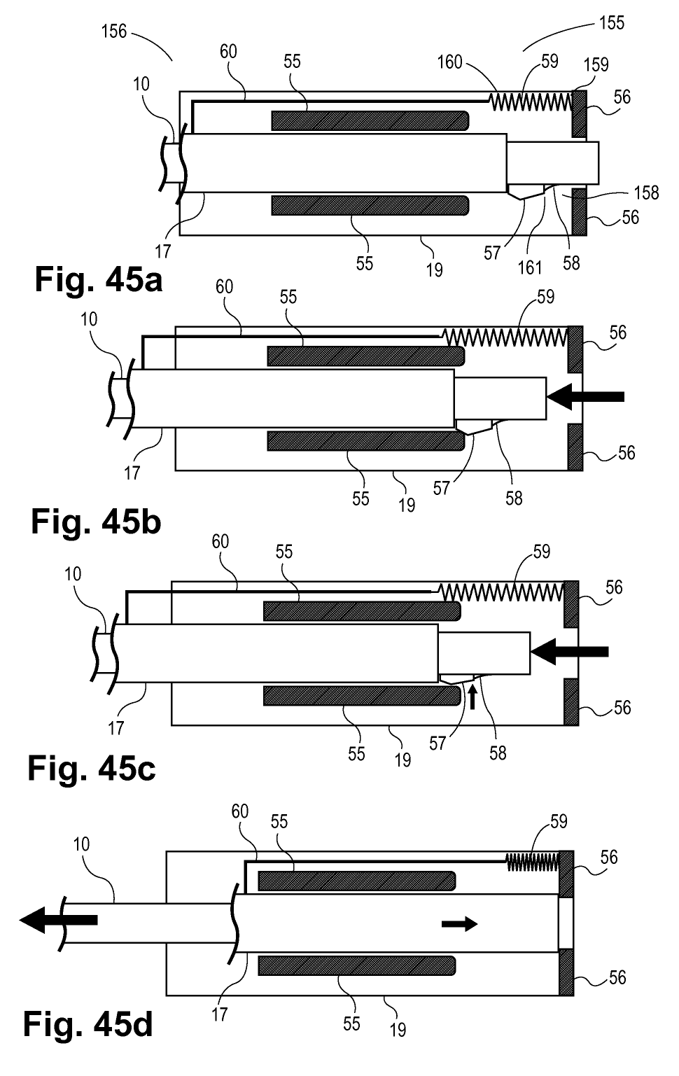

As an additional example of a handle mechanism for controlling the timing of the implant deployment, FIG. 45a depicts another embodiment of the mechanism carriage 19 with the delivery shaft body 10 and the push-tube 17 concentrically aligned, as in the previous embodiments, and slidably constrained within two ribs 55 that are rigidly attached to the mechanism carriage 19. The proximal portion 155 of the mechanism carriage 19 has two proximal blocks 56 that serve as an anchor point for proximal end 159 of the extension spring 59. The distal end 160 of the extension spring 59 is fixed to the distal portion 156 of push tube 17 by means of a tube attachment 60. A catch 57 engages with the proximal edge of the push-tube 17 and prevents the push-tube 17 from translating proximally. A flat spring 58, with the proximal end 158 attached to the proximal portion 155 of the shaft body and distal end 161 fixed to the catch 57, provides an outward bias to the catch 57 to prevent unintended release of the push-tube 17 while the extension spring 59 is under tension. A small opening in the shaft body 10 accommodates the catch 57 and allows the catch 57 and flat spring 58 to be inwardly deformed.

A force is applied to the proximal portion 155 of the shaft body 10, as shown in FIG. 45b, which could be, for example, from the handle 15 (not shown), as depicted in the previous mechanism. The force translates both the shaft body 16 and push-tube 17 simultaneously, due to the catch 57 pushing on the proximal edge of the push-tube 17, until the chamfer of the distal edge of the catch 57 just touches the proximal end of the rib 55. The extension spring 59 similarly extends further with the applied force. This portion of the mechanism cycle would represent when the two pointed penetrating tips 21 are entering the tissue, similar to the action represented in the mechanism shown in FIGS. 15a-15d.

As the applied force further translates the shaft body 10 distally, the proximal end of the rib 55 applies an inward force, depicted by the arrow, on the catch 57 which inwardly deforms the flat spring 58 and therefore causes the distal portion of the catch 57 to disengage with the proximal edge of the push-tube 17, as shown in FIG. 45c.

Subsequently, the push-tube 17 would retract proximally because of the stored energy in the extension spring 59, as the shaft body 10 continued to move distally due to the applied force, as depicted in FIG. 45d. This portion of the mechanism cycle would represent when the two pointed penetrating tips 21 had retracted from within the tissue substrate, similar to the action represented in the mechanism shown in FIGS. 16a-16d. As the shaft body 10 continued to move distally, the bioabsorbable filament implant 1 would be driven into the tissue for anchoring, similar to FIGS. 17a-17d. This alternative mechanism design provides the same sequence of movements and could be easily adapted to fit within the previous handle embodiment. Due to its concentrically aligned delivery shaft body 10 and the push-tube 17, the applied force to the mechanism is directly applied to the implant and therefore would be suitable for use with harder tissue, for example, subchondral or cortical bone.

While it is understood that the implant can be utilized during open surgery and placed directly into the target tissue or treatment site, as well as during arthroscopic surgery where the implant can be introduced into the target tissue region via an arthroscopic port, alternatively, the implant can also be introduced directly through a small incision in the skin, without the need for a port or open surgical access, and can be either be performed at the time of the initial joint surgery or even at a follow-up appointment, where the treatment can be done in a surgicenter or even in a physician exam room.

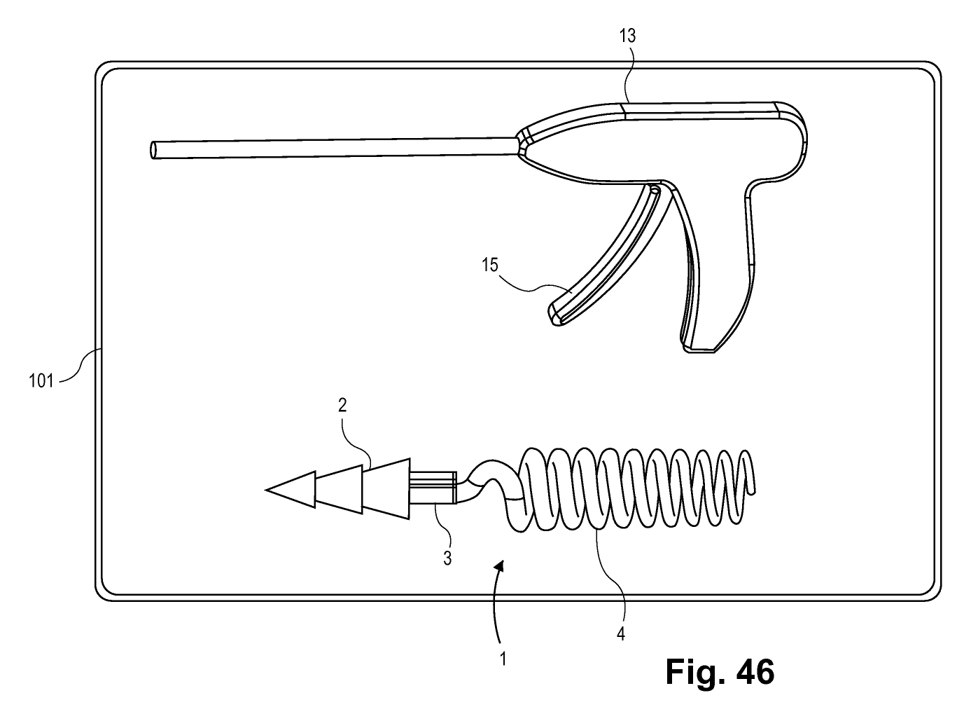

In a preferred embodiment, a kit may be provided including one or more of the components and devices described herein. The kit may include any combination of components or single components and preferably is formed by a package 101 suitable for operating room use. For instance, the package and/or individual components of the package may be hermetically sealed or be a hermetically sealed container to ensure the cleanliness of the particular component. An exemplary embodiment of the kit is shown in FIG. 46, and may include a package 101 or container having one or more bioabsorbable filament implants 1. As described herein the implant may include a tip 2, filament member 4, and interface 3 as an integral or unitary device, or as separate components which may be attached together. To this end, the package 101 may optionally include any one or more of the tip 2, interface feature 3, and elongated filament member 4. Furthermore, any one of the tips or filaments or interfaces described herein may be substituted in place of the currently illustrated exemplary embodiment shown in the kit. The package may also include a delivery tip 7 and/or delivery tool 13. While delivery tool 13 is specifically illustrated in the kit, any suitable delivery device or mechanism may be included or substituted for the exemplary embodiment shown. In a preferred embodiment, the kit includes the insertion tool 13 and one or more filaments 4 that are impregnated with medicament or alternatively one or more implants 1 all inside a package 101. Alternatively, a generic delivery tip and or tool may be provided in a separate package. As described herein, a variety of tip designs are provided for different properties. Accordingly, a variety of tips may be included in a single package or more than one package. Likewise, filaments are provided to deliver a variety of drugs or other materials as previously described. To this end, one or more filaments or bioabsorbable implants may be provided in one or more packages to provide different medicament options.

While embodiments of the present invention have been shown and described, various modifications may be made without departing from the scope of the present invention. The invention, therefore, should not be limited, except to the following claims, and their equivalents.

I claim:

1. An implantable medicament delivery device for administration of a medicament to a mammalian body comprising an implantable filament formed of a bioabsorbable material and carrying a medicament, the material being capable of eluting the medicament.

2. The delivery device of claim 1 wherein the medicament is impregnated in the filament.

3. The delivery device of claim 1 wherein the medicament is an analgesic.

4. The delivery device of claim 1 wherein the medicament is an anesthetic.

5. The delivery device of claim 1 wherein the medicament is an anti-inflammatory.

6. The delivery device of claim 1 wherein the medicament is a steroid.

7. The delivery device of claim 1 wherein the material is capable of eluting the medicament over a period of time.

8. The delivery device of claim 1 wherein the filament includes an end and an anchor at the end for localizing the filament to tissue being treated in the mammalian body.

9. The delivery device of claim 8 wherein the filament and anchor are formed as a unitary device.

10. The delivery device of claim 8 wherein the anchor includes a tapered cone.

11. The delivery device of claim 8 wherein the anchor includes a plurality of tapered cones concentrically disposed along an axis and joined in series.

12. The delivery device of claim 8 wherein the anchor includes a series of conical ridges tapering in diameter towards a tip.

13. The delivery device of claim 8 wherein the anchor includes a threaded portion.

14. The delivery device of claim 8 wherein the anchor includes a plurality of flexible tabs.

15. The delivery device of claim 14 wherein the plurality of flexible tabs extend proximally from a tapered cone in a circumferentially spaced-apart pattern.

16. The delivery device of claim 8 wherein the anchor is cannulated.

17. The delivery device of claim 1 wherein the filament has a portion formed from an elongate member having a first end and a second end and a diameter that decreases from the first end to the second end.

18. The delivery device of claim 1 wherein the filament includes a helical portion.

19. The delivery device of claim 18 wherein the helical portion has a first end and a second end and a diameter that reduces from the first end to the second end.

20. The delivery device of claim 18 wherein the helical portion is formed from an elongate member having a first end and a second end and a diameter that decreases from the first end to the second end.

21. The delivery device of claim 1 wherein the bioabsorbable material is a hydrophobic polysaccharide.

22. The delivery device of claim 1 wherein the bioabsorbable material is a hydrophilic polysaccharide.

23. The delivery device of claim 1 wherein the bioabsorbable material is selected from the group consisting of polylactic acid and polyglycoloic acid.

24. The delivery device of claim 1 wherein the bioabsorbable material is a copolymer of polylactide-co-glycolide.

25. The delivery device of claim 1 wherein the bioabsorbable material includes at least one of a radiopaque material or salt.

26. A delivery tool for use with an implantable device to treat a joint of a mammalian body comprising an elongate tubular member having a proximal end and a distal end and a passageway extending from the proximal end to the distal end, a penetration element having a sharpened tip slidably disposed in the passageway and moveable between a first position in which the tip is recessed within the distal end of the elongate tubular member and a second position in which the tip is at least partially extended from the distal end, the penetration element being adapted to carry the implantable device, and an actuation mechanism at least partially carried by the elongate member for moving the penetration element from the first position to the second and for delivering the implantation device from the elongate tubular member into an implanted position in the joint.

27. The delivery tool of claim 26 wherein the distal end of the elongate tubular member has an outer diameter is configured to be received within an arthroscopic port.

28. The delivery tool of claim 26 wherein the elongate tubular member has an exchangeable end configured for implanting a plurality of implantable devices.

29. A method of administering a medication to a mammalian body comprising implanting a filament into a joint of the mammalian body, the filament being formed of a bioabsorbable material and carrying a medicament, and eluting the medicament from the filament after placement of the filament in the joint to aid in healing of the mammalian body in the vicinity of the joint.

30. The method of claim 29 wherein the filament is implanted in an articular space.

31. The method of claim 29 wherein the medicament is an analgesic.

32. The method of claim 29 wherein the medicament is an anesthetic.

33. The method of claim 29 wherein the medicament is an anti-inflammatory agent.

34. The method of claim 29 wherein the medicament is a steroid.

35. A kit for use in post operatively treating a joint of a mammalian body comprising a package including a bioabsorbable filament carried within the package, the bioabsorbable filament carrying a medicament.

36. The kit of claim 35 further comprising a tool for delivery of the bioabsorbable filament to the joint.

37. The kit of claim 35 wherein the filament is hermetically sealed within a container.
